(19) United States
(12) Patent Application Publication
(10) Pub. No.: US 2005/0176007 A1
(43) Pub. Date: Aug. 11, 2005

(54) Discriminative analysis of clone signature

(75) Inventors: Marcel De Leeuw, Renage (FR); Jean-Francois Mouret, Coublevie (FR); Jean-Paul Issartel, Saint-Egreve (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Gemone Express, Meylan (FR)

(21) Appl. No.: 10/503,953

(22) PCT Filed: Oct. 15, 2002
(86) PCT No.: PCT/IB02/04528

Related U.S. Application Data
(60) Provisional application No. 60/356,026, filed on Feb. 11, 2002.

(51) Int. Cl. C12Q 1/68; G06F 19/00; G01N 33/48; G01N 33/50; C12P 19/34

(52) U.S. Cl. 435/6; 435/91.2; 702/20

(57) ABSTRACT

The invention relates to signatures obtained by a method for analyzing nucleic acid, wherein said nucleic acids are partially sequenced, not using all 4 chain extension terminators, a signature is generated, corresponding to the partial sequence, said signature being compared with other theoretical or actual signatures to analyze said nucleic acid.

### PRIORITY

This application is a National Phase Entry under §371 of International Application No. PCT/IB02/04528, filed Oct. 15, 2002, which in turn claims the benefit under §119(e) from U.S. Application No. 60/356,026, filed Feb. 11, 2002.

### FIELD OF THE INVENTION

The invention relates to a method for analyzing nucleic acid, wherein said nucleic acids are partially sequenced, a signature is generated, corresponding to the partial sequence after electrophoresis, said signature being compared with other theoretical or actual signatures to analyze said nucleic acid.

### BACKGROUND OF THE INVENTION

Nucleic acid sequencing is performed according to the well-known method of Sanger et al (enzymatic chain elongation reaction), in which a primer is designed to hybridize the test nucleic acid molecule, chain elongation is performed by enzymatic addition of deoxyribonucleotides (dNTPs), which will stop when a terminator dideoxyribonucleotide (ddNTP), present in the reaction pool, is added in place of the corresponding dNTP. A mixture of differentially extended molecules is created, which is resolved by electrophoresis, generally on polyacrylamide gels.

Most of the times, detection of the differentially sized molecules is performed using a signal emitted by these molecules, the primer or the ddNTPs being labeled. When the ddNTPs are labeled, four different labels can be used (fluorescent labels), the elongation reaction can be performed in a single reaction, and electrophoresis of the whole mixture can be performed in a single lane of the denaturing electrophoretic gel, using different filters to detect each label. Applied Biosystems or Amersham for example have developed sequencing machines to perform such sequencing run. In high throughput systems, the migration of the sequencing reaction is performed in a capillary.

When the label is not carried by the ddNTPs, a set of four primers is generated by independent labeling with four different labels and each specifically labeled primer is used with a given ddNTP in an elongation reaction. Thus, four reactions are performed, in which one labeled primer and one ddNTP is used. The four reactions are then pooled, and the reaction products are resolved on the electrophoresis gel, using different filters to detect each label corresponding to each primer and consequently to the ddNTP used.

Other possibilities are used for sequencing nucleic acid molecules. The two above described strategies rely on fluorescent labeling and detection. One can also use radioactive labeling. It is generally less interesting than fluorescent labeling, where different labels can be used, allowing the performance of only one reaction, or the pooling of the reaction mixtures, before detection. When radioactive labeling is used, it is usually necessary to perform four reactions (one for each ddNTP), and to load four lines on the electrophoresis gel. Thus, the use of fluorescent labels is usually preferred for high throughput sequencing.

Another possibility can be used for sequencing nucleic acid, in particular mass spectrometry. With this technique, the differentially sized fragments obtained after the elongation reaction are separated according to their mass, using a mass spectrometer and known techniques. The obtained representation is a “ladder” of peaks, and the mass difference between two peaks leads to the determination of the added nucleotide (A: 312.2 Da; T: 301.2 Da; G: 328.2 Da; C: 288.2 Da). The data can be output as peaks similar to a sequencing electropherogram and/or as tabular data for importing into a spreadsheet.

Data from analysis of DNA sequence reactions based on fluorescent labeling which are derived from sequencing machines are available in the form of electropherograms consisting of series of peaks resulting from the detection of optical signals which are characteristic of the DNA sequences analyzed. These electrophoretic traces are usually exploited in two different ways:
1) to determine the composition of a DNA fragment, i.e. decipher the sequence of a DNA fragment (DNA sequencing). Operation based on attributing to each of the electropherogram peaks a base name (from a range of 4 bases: A, G, C or T making up the DNA fragment) (base calling). Base calling is made possible because all four terminator dideoxyribonucleotides or primers are labeled by fluorescent groups with specific optical features.
2) to determine the size of the fragments, in number of bases, originating from enzymatic digestions (restriction fragment length analysis) or from PCR reactions.

These two specific exploitations of the electrophoretic traces use different analytical strategies and programs. In both cases, the analysis imposes considerable constraints on the resolution of the peaks and the rate of migration of the samples, and on the coherence of migration of the fragments in the electrophoresis capillaries as a function of their size. These constraints increase the duration of processing the samples and limit the throughput of the machine. In addition, not insignificant “contextual” effects due to the sequence, itself, of the DNA fragments analyzed result in aberrations in the height of the peaks or their spectral composition. These aberrations constitute serious problems which affect the validity of base calling. Another problem which seriously harms the quality of the base calling lies in an artifact of electrophoretic mobility of the fragments which leads to “shifting” of the peaks on the electrophoresis trace. This mobility artifact results from the acceleration or from the slowing down of certain fragments in the course of their electrophoretic migration. It reflects the fact that the rate of migration of a DNA fragment in the matrix of the electrophoresis capillary is not strictly related to its size but is influenced by particular DNA structures (particular folding of the DNA fragment).

Nevertheless, some sequencing machines, such as the ABI 3700 sequencer machine is such that the above mentioned migration artifacts are negligible and, which facilitates the base calling, as the peaks corresponding to each nucleotides are evenly spaced.

Base calling may also be performed when mass spectrometry is used, as the distance between the different obtained peaks corresponds to the mass of the incorporated nucleotide.

Base calling makes it possible to obtain an alphanumerical sequence (in particular using the symbols A, T, C, G) from the analogical curve (electropherogram or mass spectrometry graph).

Characteristic information-containing data of a particular DNA fragment, or clone, is required in order to unambiguously identify a clone within a collection composed of multiple clones making up a DNA library.

Two types of libraries exist: libraries composed of cDNAs cloned into vectors and libraries made up of individual genomic DNA fragments also cloned into vectors.
cDNA libraries are obtained from gene expression products (gene transcripts or mRNAs) and, as a result, may provide qualitative data by revealing which genes are expressed in a cell or a tissue, and quantitative data relating to the level of expression of each of the genes. Because of this, cDNA libraries constitute a resource which is full of potential in the field of functional genomics.
The second type of library corresponds to libraries of genomic DNA fragments and to collections of PCR fragments obtained by targeted amplifications of regions of a genome. These libraries are in particular used in the context of projects aimed at sequencing whole genomes, or parts of genomes, of diverse organisms. Other libraries which are of considerable scientific and economic value correspond to sequence databases which collect annotated genome sequence information, and polymorphism databases (SNP database). Overall, any sequencing of DNA fragments makes it possible to constitute sequence databases which, as a result, represent reference databases which can be used for subsequent analyses in silico (referential database).

A sequence of limited size corresponding to a fraction of the total sequence of a DNA clone may constitute a characteristic and specific signature of a clone, allowing it to be identified in a set of signatures (this signature may be called a tag). This observation is the basis of methods for exploring and analyzing the transcriptome of cells or tissues, such as the SAGE method (Serial Analysis of Gene Expression: Velculescu etal, Science. 1995;270(5235) :484-7, also described in U.S. Pat. No. 5,695,937) or the MPSS method (Massively Parallel Signature Sequencing: Brenner et al, Nat Biotechnol. 2000;18 (6):630-4).

A third approach which can be exploited for recognizing a transcript uses an oligonucleotide (in general less than 70 bases) which is representative of a limited portion of the DNA to be detected and which is used as a hybridization probe (oligo arrays). Only approaches linked directly to the analysis of fragments by sequencing will be explained in detail here.

Three principles underlie the SAGE methodology: 1) A short sequence tag (10-14 bp) contains sufficient information to uniquely identify a transcript provided that the tag is obtained from a unique position within each transcript; 2) Sequence tags can be linked together to long serial molecules that can be cloned and sequenced; and 3) Appraisal of the number of times a particular tag is observed provides the expression level of the corresponding transcript.

In the MPSS method, a planar array of a million template-containing microbeads in a flow cell at a density greater than 3×10⁶ microbeads/cm² is assembled. Sequences of the free ends of the cloned templates on each microbead are simultaneously analyzed using a fluorescence-based signature sequencing method that does not require DNA fragment separation. Signature sequences of 16- 20 bases are obtained by repeated cycles of enzymatic cleavage with a type IIs restriction endonuclease, adaptor ligation, and sequence interrogation by encoded hybridization probes.

In the case of the SAGE technique, a certain number of drawbacks specific to the method exist, in particular the multiple and complex steps for preparing the samples. In addition, the SAGE technique leads to the creation of a sequencing matrix which multimerizes the signatures of the cDNAs in the form of tags of about ten bases. As a result, although the creation of this matrix is very suitable for sequencing, in series, several signatures per matrix, at the end of the analytical process, only the multi-tag containing matrices remain available. The individual cDNA clones corresponding to the different tags have never been generated through the SAGE process. As a consequence, more thorough study of the physicochemical and biological cDNA parameters (size, sequence, identification, detection of the protein-coding region, etc. ) is not straight forwardly possible and can be carried out only after having recreated, independently, a new cDNA library containing whole cDNAs and no longer only tags of about ten bases. The SAGE method indeed destroys the cDNAs when creating the tags.

With both SAGE and MPSS methods, only short sequence tags are usually generated. The short size of these tags may severely impair the accuracy of the identification of the corresponding gene transcripts.

### SUMMARY OF THE INVENTION

The present invention is based on the concept that it is interesting to develop a method for analyzing nucleic acids, especially for analyzing DNA libraries, at reduced cost, with the output giving enough information about the nature of said DNA. The inventors have determined that it is not necessary to fully sequence a DNA piece, with four reaction terminators (stopping the extension at each of the four bases A, T, C, G), but that a “partial” sequencing reaction, with less than four reaction terminators gives a usable information.

This piece of information, which is highly specific on any DNA fragment is called a signature.

A characteristic signature of the DNA may be obtained, using the methods described in the invention, in some embodiments. Characteristic signatures according to the invention can be generated from genomic DNA libraries or cDNA libraries.

The characteristic signature of the DNA may be a fraction of the sequence of this clone. It may also consist of a fragment of the electropherogram derived from sequencing machines. The signature is, in this case, an element which can be reproduced in the form of a graph composed of several successive peaks of variable heights (NB: the methods of representation of this signature are not, however, restricted to simply representing a graph). The signature may also be a fragment of the “ladder” obtained after mass spectrometry analysis, which can also be represented as a graph.

The series of consecutive peaks extracted from a fragment of the sequence of a DNA clone, to which quantitative parameters which reflect the characteristics of this series of peaks can be associated, constitutes a specific signature. The information associated with the signature is not necessarily exhaustive, in the sense that it does not necessarily fully reveal the sequence of the 4 bases which make up the clone analyzed or the fraction of sequence which defines the signature, but it is sufficiently rich to confer specificity on it and to guarantee the efficiency of discrimination of the clones of a library, based on the comparison of their signatures. The signature therefore constitutes, more generally, a symbolic representation of the electrophoresis or mass spectrometry trace.

The aim of the method according to the invention is to be able to analyze the overall nature of a DNA fragment library by creating signatures specific for every single clones which make up this library using an optimized method for sequencing cloned DNA fragments.

The method of the invention may be referred as DACS (Discriminative Analysis of Clone Signature).

According to still another aspect, the present invention provides a method for generating graphical data representative of the similarity of first and second x-coherent analog signatures of nucleic acid molecules, comprising the steps of:
dividing each signature into a plurality of fragments,
performing a cross-correlation of each fragment of the first signature with each fragment of the second signature, respectively, so as to generate a matrix of cross-correlation values,
generating a matrix of graphical zones for display, wherein each zone has a visual property determined from a corresponding cross-correlation value, and
displaying said matrix.

Preferred aspects of this method are as follows:
the fragments are overlapping.
each signal comprises individual peaks, and each fragment contains a plurality of peaks, preferably from 10 to 20 peaks.
each visual property is selected from a color or a grayscale level.
a clearer color or grayscale value corresponds to a higher correlation between fragments.

### BRIEF DESCRIPTION OF THE DRAWINGS

FIG. 1 is a schematic representation of an embodiment of the method of the invention. Partial sequencing according to the invention is performed on different clones, using one chain extension terminator. 4 clones, each labeled with a different dye are pooled and the mixture is analyzed by capillary electrophoresis in a DNA sequencer such as ABI 3700. In this embodiment, three pools are serially injected in each capillary. The obtained chromatograms are then analyzed to perform a demultiplexing according to the different labels, suppress the portion corresponding to the vector carrying the clones, and obtain the signatures according to the invention.

FIG. 2 corresponds to a representative signature of the invention obtained with the method of the invention, using ddC as the chain terminator.

FIG. 3 illustrates examples of signatures that can be generated for one specific clone, by using all four different reaction mixtures containing individual chain extension terminator. It is seen that definition of a threshold makes it possible to assign a peak above said threshold to the corresponding base.

FIG. 4A (bottom part) is the comparison of the chromatogram obtained for a sequence using the method of the invention with only one chain extension terminator (ddTP in this case, top curve). The output from the sequencer is analyzed by the Sequencher™ software. The bottom curve shows a chromatogram obtained on the same sequence, using the four chain extension terminators.

FIG. 4B (top part): the signature obtained by the method of the invention is compared with the base-called sequence obtained by sequencing with the four chain extension terminators.

FIG. 5 represents a chromatogram obtained with the Taq polymerase, as compared to the theoretical curve (reference).

FIGS. 6 to 9 represent schematic summaries of different preferred strategies to perform the invention.

FIG. 10 represents an energy diagram obtained when cross-correlating two signatures.

FIG. 11 represents a correlogram obtained after comparing two signatures (clone 1 and clone 2). The signatures were compared by cross-correlation of short fragments, as described in the specification. part A: no correspondence between the signatures, which represent two different DNA sequences. Part B: correspondence between the signatures which represent two similar DNA sequences.

FIG. 12 represents in greater detail the approach used to generate the correlogram of FIG. 11.

### DETAILED DESCRIPTION OF THE INVENTION

The invention relates to a discriminative, reproducible, x-coherent signature from a nucleic acid molecule, which is preferably unknown a priori, obtained by a method comprising the steps of:
a) performing an enzymatic chain elongation reaction on said nucleic acid molecule, using at least one primer to start the elongation process, and at least one chain extension terminator, whereas not all four possible terminators are used (partial sequencing reaction), in order to obtain differentially sized products, wherein said differentially sized products can be evidenced by a detectable signal,
b) separating the differentially sized products obtained in step a),
c) detecting the detectable signals corresponding to each of the differentially sized reaction products after separation, and
d) creating the signature directly or indirectly based on the detected signals in step c).

In the following invention, “partial sequencing reaction” is intended to mean a sequencing reaction performed by the enzymatic chain elongation method, when not all four terminators are used.

In a preferred embodiment, separation of said differentially sized products is performed by electrophoresis. In another embodiment, separation of said differentially sized products is performed by mass spectrometry, and the “detectable signal” is the mass of the differentially sized products.

By “chain extension terminator”, it is meant a compound that can be enzymatically incorporated in the nucleic acid chain formed during the sequencing reaction, that is base specific (a chain extension terminator according to the invention can not be randomly incorporated), and that forbids further chain extension after its incorporation.

This signature, according to the invention is reproducible, which means that the analysis of the same DNA fragment with the same method will give the same signature.

The signature is also discriminative, which means that two different signatures correspond to two different DNA molecules.

The signature is also x-coherent, which means that at any point on the x-axis, the distance between two information elements (peaks) is at least approximately correlated to the distance between the elements generating this information in the starting nucleic acid molecule.

For example, this means that the distance between two electropherogram peaks corresponding to the base “A” is correlated to the numbers of nucleotides between the two bases “A” in the molecule. This may be obtained for instance by using the ABI 3700 (Perkin-Elmer Applied Biosystems Inc) sequencer.

When mass spectrometry is used for the definition of the signature, the distance between two mass peaks is directly correlated to the mass existing between the two nucleotides on the starting sequence.

In a preferred embodiment, exactly one chain extension terminator is used.

In another embodiment, exactly two chain extension terminators are used.

In yet another embodiment, exactly three chain extension terminators are used.

In a preferred embodiment, said base-specific chain extension terminator is a dideoxyribonucleotide, in particular chosen in the group consisting in ddA, ddT, ddC, ddG. In another embodiment, said base-specific terminator is a morpholino, as described in particular in patent applications FR 2 790 004 A, FR 2 790 005 A or WO 00/50626 A.

In an embodiment, exactly two chain extension terminators are used, in particular chosen in the group consisting in (ddA and ddT), (ddA and ddC), (ddA and ddG), (ddC and ddT), (ddC and ddG), and (ddT and ddG).

In an embodiment, exactly three chain extension terminators are used, in particular chosen in the group consisting in (ddA, ddC, ddG), (ddA, ddC, ddT), (ddA, ddT, ddG), and (ddC, ddT, ddG).

In a specific embodiment, step c) is performed by the step of creating a graph (electropherogram from electrophoresis or mass spectrometry output), based on the detected signals in step c), which can be used as the signature.

In particular, said signature comprises the intensity of said signals and/or the time lapse between two consecutive signals.

In one embodiment, the signature according to the invention is a string of alphanumeric signs, with a specific sign for the bases that are determined (using the terminator) and that correspond to a specific nucleotide, and a “joker” sign for the other bases.

As a way of illustration, if ddA is used as the terminator for the sequence ATCAAGGCATTAGT, the signature may be a string as follows: ANNAANNNANNANN.

The signature corresponding to the sequence of a DNA fragment, comprising a sequence obtained by sequence reaction of said DNA fragment wherein not all four possible terminators are used, is an object of the invention, and in particular the signature consisting of a sequence obtained by sequence reaction of said DNA fragment wherein not all four possible terminators are used.

In a preferred embodiment, said enzymatic chain elongation reaction of step a) is performed on a limited number of bases of said nucleic acid molecule, for example about 50,100, 150,200 or 250 bases. The number of bases is the maximum length of the chain elongation product, and the person skilled in the art is able to determine the reaction conditions in order to obtain the desired length. The desired length may depend on the nucleic acid that is analyzed, and on the level of precision that is needed. It is reminded that a sequence of about 20-25 nucleotides is considered as defining a unique cDNA. With the method of the invention, where the whole sequence is not available, it is expected that about partially sequencing 100 bases is enough to obtain the signature according to the invention corresponding to the nucleic acid.

In the most preferred embodiment, said electropherogram and/or said alphanumerical signature is created by means of computer-assisted “basecalling”. In a specific embodiment, said computer-assisted “basecalling” is performed with the Sequencher software (Gene Codes Corporation).

The basecalling consists in allocating a specific alphanumerical character to an informative element of the signature (peak in the electropherogram, corresponding to the detected nucleotide), and a string of “joker characters” corresponding to the nucleotides that are located between two consecutive detected nucleotides.

For base calling of a signature from an electropherogram, a peak intensity threshold is decided, and the nature of the base is then determined for all the peaks the intensity of which is above the threshold, where the “joker” sign is determined for the peaks the intensity of which is below the threshold.

When basecall is performed from a signature obtained from mass spectrometry, the number of bases between two peaks is determined by solving the equation in x, y and z:

ΔM−Mdn=x*Mn1+y*Mn2+z*Mn3, with x, y, and z being integers, where AM is the difference of mass between two peaks; Mdn is the mass of the determined nucleotide (for which the dideoxynucleotide has been used); Mn1 is the mass of one of the non-determined nucleotide, Mn2 is the mass of another non-determined nucleotide, and Mn3 is the mass of the last non-determined nucleotide. Due to the differences between masses of the nucleotides, this equation has a unique solution.

The number of “joker” signs between the two determined nucleotides is then x+y+z.

In an embodiment, said detectable signal that will eventually be on the extension products is carried by a label on said primer.

In another embodiment, said detectable signal is carried by a label on said chain extension terminator.

In the preferred embodiment, said label is a fluorescent label. In another embodiment, said label is a radioactive label.

In a preferred embodiment, said electrophoresis is a capillary electrophoresis. In another embodiment, said electrophoresis is performed on a classic polyacrylamide electrophoresis denaturing gel, in particular in an apparatus such as ABI PRISM 377 DNA Sequencer (Applied Biosystems Inc), ABI 3700 DNA sequencer or the like.

Due to an advantage of the method for generating the signatures according to the invention, in a particular embodiment, it is possible to perform the electrophoresis and detection steps on multiple samples (for example 4 samples) at the same time.

In an embodiment, one performs multiple “partial sequencing” reactions, using one chain extension terminator for each reaction, said terminator being differentially labeled for each reaction (for example fluorescently labeled, red for the first reaction, blue for the second, green for the third and yellow for the fourth). The reaction mixtures are then pooled and the products are resolved by electrophoresis. Simultaneous detection is performed for each of the four labels, using suitable filters, and the obtained signals are demultiplexed in order to obtain the signatures for the nucleic acid in each of the samples.

In another embodiment, one performs the partial sequencing reaction on different clones, using more than one (not all four) chain extension terminator identically labeled in each sequencing mixture, but differentially labeled between two different sequencing mixtures. The mixtures are then pooled and analyzed.

In another embodiment, one performs the partial sequencing reaction on different clones, using a differentially labeled primer for each clone, and one or more non labeled chain extension terminators (not all four), before pooling the reaction mixtures and analyzing them.

In another embodiment, four partial sequencing reactions are performed in the same mixture, on four clones, using four differently labeled primers, that are each specific of one clone, and one (or more, but not all four) non labeled chain extension terminator. The reaction mixture is directly analyzed by electrophoresis.

Thus, the invention also makes use of a method, wherein electrophoresis and signal detection steps are simultaneously performed on multiple nucleic acid molecules subjected to step a), wherein said detectable signal corresponds to the chain elongation products is different for each of the multiple nucleic acid molecules.

In a preferred embodiment, said detection step comprises the step of detecting, recording the signals and distinguishing (demultiplexing) the signals specific to each of the multiple nucleic acid molecules (as different labels are used for each clone).

Preferably, said demultiplexing is computer-assisted. The methods for demultiplexing are known in the art and are used in pieces of software of the art.

In some embodiments, said nucleic acid is further characterized by the comparison of its signature with a database of signatures.

Note that two signatures according to the invention can be compared by ways of subtraction using software such as the Synquence™ software described in U.S. Pat. No. 6,195,449, the content of which is incorporated herein by reference.

The database of signatures is either the database obtained after performing the method of the invention to a library of clones, or a theoretical database, obtained by using the available DNA databases (GenBank, EMBL), and optionally transforming the sequences present in these databases (consisting of strings of letters, using the A, T, C, G symbols) to strings of letters wherein only remains the actual ddNTP(s) used in the method of the invention to obtain the test signature. This modification is only optional and the alphanumerical signature can directly be compared to the actual databases.

Thus, it is possible to compare either the analog signature (obtained directly after performing the method describes above) or the alphanumerical signature (pseudo-sequence) obtained after base calling (or pseudo-base calling).

Different methods for comparison of the pseudo-sequence of a nucleic acid with a database of sequences or pseudo-sequences can be used, based on the methods similar to the local homology algorithm of Smith and Waterman, Ad. App. Math 2: 482 (1981), the homology alignment algorithm of Neddleman and Wunsch, J. Mol. Biol. 48:443 (1970), the search for similarity method of Pearson and Lipman, Proc. Natl. Acad. Sci. (U.S.A.) 85:2444 (1988), computerized implementation of these algorithms (GAP, BESTFIT, BLAST N, BLAST P, FASTA, and TFASTA in the Wisconsin Genetics Software Package, Genetics Computer Group (GCG), 575 Science Dr., Madison, Wis.), or inspection.

In particular, one can use BLAST (Altschul et al) or LocalAlign (implementation of Smith and Waterman) functions that are well known in the art, using the penalty matrix as below (when the detected nucleotide is “A”).

| | A | C | G | T | N |
|---|---|---|---|---|---|
| A | 8 | | | | |
| C | −8 | 2 | | | |
| G | −8 | 2 | 2 | | |
| T | −8 | 2 | 2 | 2 | |
| N | −8 | 2 | 2 | 2 | 2 |

This matrix makes it possible to allocate a score of 8 to each A-A alignment, a score of 2 to each [CGTN]-[CGTN] alignment, and apenaltyof-8 to each A-[CGTN] mismatch.

In a preferred embodiment, the comparison is performed on analog signals by cross-correlation Rxy, between said signal x and another signal y in the database of signatures.

In particular, said cross-correlation consists, for all possible alignments of two analog signals (i.e. shifted from each other through a variable step t), in calculating the cross-correlation or energy value R_{xy} (t) in the following manner:

$${R}_{xy} ( t ) = x ( t ) ⊗ y ( t ) = {\int }_{- ∞}^{+ ∞} x ( τ ) y ( t + τ ) ⅆ τ$$

and in recording the results for each alignment.

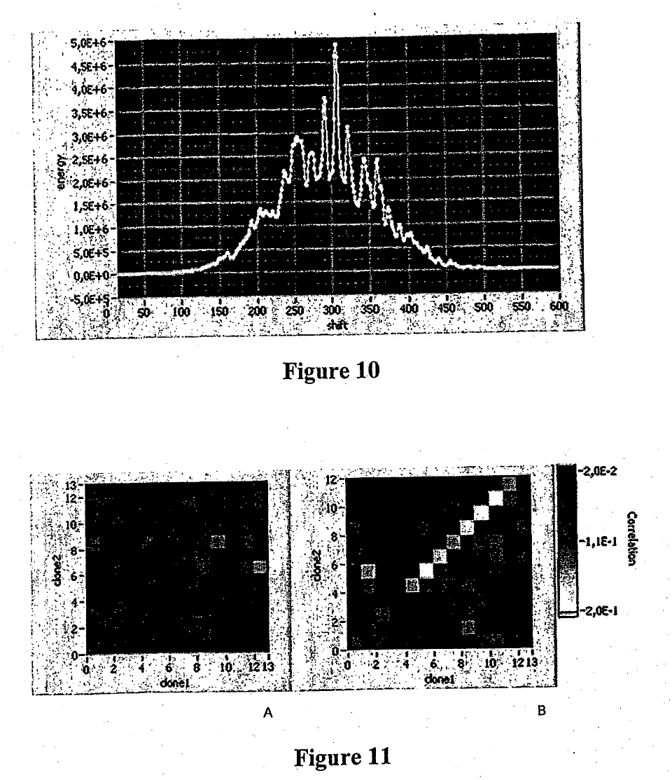

This calculation thus returns an estimation of the correspondence for every step of shift, which can itself be shown as an analog signal. FIG. 10 illustrates an example of such cross-correlation signal of two signatures, where the Ry (t) value as a function of the shift value t is represented.

In this signal, the interesting feature is the localization of the maximum and in its amplitude, respectively corresponding to the optimal alignment and the relevance of this alignment. In other words, a strong correlation will be revealing by a major peak as shown in the central portion of FIG. 10.

The gap step T represents the degree of fineness with which Rxy (t) is computer, and can be varied according to the desired accuracy of the cross-correlation and to the computing capacity and available memory of the computer system running the cross-correlation. This gap step may be fixed for instance to 10 points by peak.

In addition, the calculation can in practice be locally performed on different fragments of each of the overall signatures to be compared, for several reasons:
the coverage of the signature may be too important for making the difference between a global correspondence which is not very good and a local correspondence which is good but which concerns only a segment of the signature (case of two clones with partial overlapping),
if the number of samples per peak is not strictly identical between two signatures, the method will return a bad score, while it may be two signatures of the same clone, realized in two different capillaries during one run of electrophoresis (the impact of small variations in this spacing is proportional to the length of the signature, so that cutting the signature in fragments is an advantage);
the calculation of the correlation by a conventional computer system can be performed faster if the totality of the points of both signatures can be put in the cache storage of the processor (cutting the signal in fragments and to reconstructing the puzzle later can thus be advantageous in term of computation).

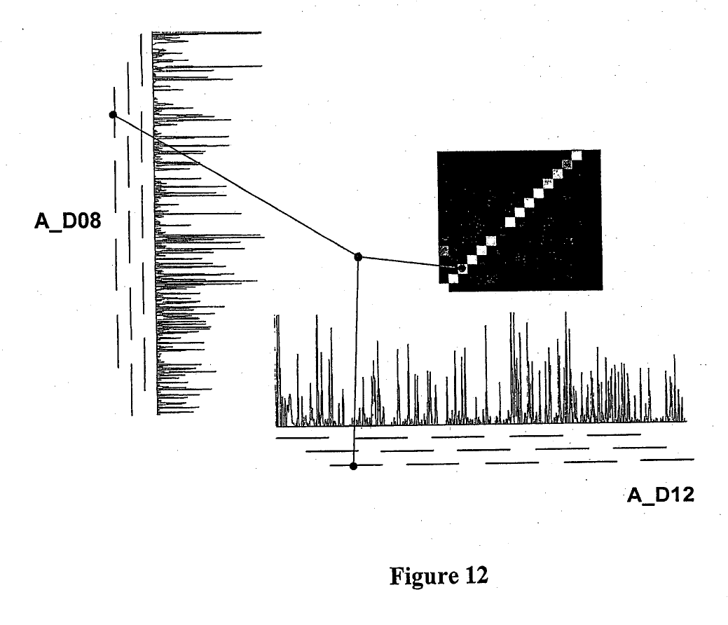

FIG. 12 illustrates the process of using the cross-correlations computed for fragments of the starting signals to generate, a so-called “correlogram” allowing to visually determining useful information concerning the two signatures.

From a practical standpoint, the two analog signatures to be cross-correlated are digitally encoded at suitable time and amplitude resolution. For instance, the encoding can by done using 16 bit words and a time resolution of 10 to 50 samples per peak interval of the signal. Typically, the time resolution is identical to the gapstep T used for shifting the signals relative to each other.

The cross-correlation can thus be performed by digital computation using the value of each sample.

As shown in FIG. 12, each signal is subdivided into a plurality of fragments overlapping each other (preferably around 50% overlapping rate). Each window has a length corresponding to a number of peak intervals sufficient to generate a reliable cross-correlation. For instance, each window covers 10 to 20 peaks.

The cross-correlation process then computes the cross-correlation value for each pair of windows respectively belonging to the two analog signatures, so as to generate a two-dimensional matrix of values. For display purposes, these values are converted into color or greyscale values, with a depth of 16 or 256 values or even more, which are reflected in the correlogram defined by the matrix of squares shown in FIGS. 11 and 12. Preferably, the higher the correlation between the starting signals, the clearer the color or gray level shown in the correlogram.

It can easily be understood that an alignment of clear squares in a diagonal direction reflects a high degree of similarity between both signatures. If such alignment is located on the main diagonal of the matrix, then this reflects that the signatures are similar from a common starting point, i.e. aligned. If on the contrary such alignment is shifted away from said main diagonal, then this reflects that the signatures are similar but start from a different starting point (misalignment). In addition, the length of a diagonal alignment of clear squares gives an indication of the length on which both signatures are similar.

As a consequence, the mere visual observation of the correlogram can therefore give very useful indications about the two clones:
perfect diagonal (as shown in part B of FIG. 11): the two signatures are similar on a significant part of their length, so that a decision to cluster the two clones in one same group is appropriate,
no diagonal: the two clones are different, and carry different DN (as shown in part A of FIG. 11),
partial and/or shifted diagonals: at least one part of the signature of one clone correlates with at least one part of the signature of the other clone: this may give information about repeated sequences in the library (repeated sequences in the organism for a genomic library), about alternative splicing (for a cDNA library), about chimerism (when YAC or BAC are studied), about clone overlapping, etc. Such an observation means that the two clones carry identical DNA where the signatures correlate.

In a specific embodiment, the analysis carried out in the context of the method according to the invention is both qualitative and quantitative, i.e. it allows the various species of clones which make up the library to be identified and also makes it possible to quantify the representation of each one of these clones among the population of the different clones which make up the library.

When a cDNA library is created, if the library has been produced using protocols which do not bias the representativeness of each of the clones, the quantitative data directly reflect the gene expression profile of a cell or of a tissue. The data provide information on the identity of the genes which are expressed and on the rate of transcription of these genes.

In order to carry out this analysis, the method developed by the inventors can make use of the following steps:
Creating a library.
Isolating the clones.
Randomly selecting a large number of clones.
Purifying the vectors containing the cloned DNA fragments.
Sequencing each clone via an enzymatic chain termination reaction (Sanger et al, Proc Natl Acad Sci U S A. December 1977; 74 (12): 5463-7). The sequencing reaction here comprises the particularity of leading to “partial sequencing”. It is carried out using only one chain extension terminator (and not the four possible terminators) and the length of the DNA fragments created by polymerization during the sequencing reaction does not exceed a basic number defined as a function of the objective sought during the analysis (200 bases for the analysis of cDNA libraries for example). The reaction products from sequencing a clone are fluorescently labeled (on the priming primer or on the chain extension terminator). A single fluorescent compound is used for the reaction from partial sequencing of a clone.
One variant envisaged consists in constructing one or more primers for initiating the sequence reaction at multiple sites of the insert, so as to obtain a more discriminating signature. This increase in selectivity represents a considerable asset for discriminating transcripts derived from the alternative splicing of certain genes or from the expression of gene isoforms. In particular it is possible to design primers to initiate the sequencing reaction from the 3′ and 5′ end of the nucleic acid sequence. This is particularly interesting when said sequence is cloned in a vector, where the primers can be chosen within the vector. When such a strategy is used, the sequencing reaction can be performed in the same mixture with the two primers, that can be identically labeled or not.
Mixing four sequence reaction products originating from the analysis of 4 different clones. Each of the four reactions has been carried out with a different fluorescent label. The existence of fluorochromes which have very specific spectral properties, and the optical capacities of the sequencing machines, make it possible to analyze, individually, the fluorescence signals from each of the sequence reactions, even if the samples have been mixed.
Analyzing the reaction mixtures by electrophoresis.
Detecting, recording and distinguishing the signals specific to each of the reactions.
Analyzing the data for coherence-incoherence (quality control for the electrophoresis).
Creating the specific signature of each clone based on the analysis of the electropherograms.
Comparing the signatures to one another or comparing to one (or more) reference database (s).
Quantifying the representation of each clone relative to all of the detected signatures of the library.
Or
Detecting the clones having a content not represented in the reference database (s).

As the procedure according to the invention does not destroy the clones, it is immediately possible to relate a specific signature to a specific clone (nucleic acid in a vector within the library) and to go back to said clone to better sequence it if further characterization is needed (four-base sequencing reaction and complete sequencing of the whole cDNA fragment).

Thus, it is important to specify that definitive characterization of the clones by thorough sequencing is possible in the approach described in this document, since the cDNA clones are created and stored in the clone library and remain accessible for analysis. It is also possible to sort the clones which have the same signature but which are thought to originate from alternative transcripts or from the expression of gene isoforms. Specifically, it is possible to subject all the clones having the same signature to a structural analysis by enzymatic digestion followed by electrophoretic migration on a gel (RFLP analysis-restriction fragment length polymorphism analysis). This analysis allows the cDNA clones to be classified in various groups which show the same electrophoretic migration profile. At the end of this analysis, a clone from each of the groups can be thoroughly sequenced.

The sequence of the clones will make it possible to recognize the alternative transcripts of the same gene by revealing the differences relating to the alternative splicing phenomenon, and will make it possible to recognize the transcripts derived from gene isoforms.

A certain number of strong points of the method are described succinctly below.

Like that which was initiated by Okubo et al. in 1992 (Nat. Genet. 2, 173-179), the sequencing of multiple clones selected randomly from a cDNA library makes it possible to define which genes are expressed in a tissue and to estimate the level of expression of these genes (which genes are transcribed abundantly, and the transcripts of which genes are relatively uncommon in the set of cellular mRNAs).

In the method which we describe, we have continued this concept and have taken it to a level of effectiveness and reliability which, to our knowledge, has never been described. First of all, the method based on a strategy of creating libraries and sequencing the clones of this library offers major advantages compared to the previously described SAGE and MPSS methods:
the constitution of DNA libraries is a substantial process, which is part of the conventional panoply of experimental protocols in laboratories and which is generally readily mastered in molecular and cell biology laboratories. On the other hand, the construction of the libraries essential for carrying out the SAGE method is a process entailing a great deal of work, which requires a certain expertise and is the subject of a protection by a patent which forces the non-academic research scientist to pay heavy exploitation fees.
once constituted, the basic biological resource (the content of the libraries) is perpetuated and the clones remain, without modification, at the disposal of the research scientist in a purified and completely classified form. The resource therefore remains available for other additional experiments (production of PCR products, constitution of arrays, etc.). In the case of the SAGE techniques, the cDNAs constituted are destroyed since they are enzymatically reduced in the form of small tags of about ten bases in length. The whole cDNA clone is no longer available and, as a result, it is not possible to obtain any more sequence information relating to this clone other than the 9/10 bases detected. The same problem also exists in the MPSS technique. Additional experiments on the clones identified as being of interest therefore require reconstitution of the resource.

Since the partial sequencing reaction carried out in the method of the invention is intended, overall, to create a signal which corresponds not to all bases, and preferably to only one base among the four which make up DNA, the material and reagents consumed during these reactions are decreased. This makes it possible to make substantial savings given the high cost of the chemical reagents which make up the composition of sequencing reaction kits.

In the chapter regarding production costs, it may be noted that the method has been developed in order to reduce these to a maximum. It is, for example, envisaged to use terminators and enzymes which are not conventionally used in sequencing reactions because they are not compatible with the production of a conventional and complete sequencing reaction intended to completely identify the sequence of the 4 bases of a DNA fragment, but which may be perfectly suitable for producing a signature of quality by partial sequencing. These reagents, which are of rigorous quality, are nevertheless associated with greatly decreased costs of use.

Thus and in a specific embodiment, the method of the invention is performed with the Taq polymerase as the enzyme for enzymatic chain elongation.

In another embodiment, Sequenase is used, which can be obtained from United States Biochemicals.

The fact that only partial sequencing reactions is performed makes it possible to mix 4 different sequencing reactions (multiplexing) in the analysis process, for which different labels have been used. As a result, electrophoresis of this mixture in a single capillary allows 4 clones to be analyzed simultaneously.

It is also possible to perform multiloading of the pools in the single capillary lane, by serially injecting pools of reaction, as described in greater detail in Example 3 here below.

Finally, the possibility of multiloading leads to sequencing rates being increased, while at the same time limiting production costs. The accumulation of these optimization elements allows massive sequencing of several thousands of clones per library to be carried out in a limited time and at reasonable expense. It is theoretically possible to estimate that sequencing 30 000 different clones for a cDNA library should make it possible to have at least one copy of the signature of the rarest RNAs in a human cell. Partial sequencing, according to the method, of 30 000 different clones may be carried out in less than 24 hours with one sequencing machine comprising 384 capillaries. It should be noted that the duration of electrophoresis can also be considerably decreased compared to the conventional electrophoresis required for sequencing DNA fragments. In fact, the reading length for the electropherogram may be limited to 200 bases, without being prejudicial to the informative nature of the signature. This maximum length is approximately 5 times shorter than the conventional reading length for a conventional sequencing reaction. In addition, the electrophoresis conditions can be modified such that the migration is very greatly accelerated (by increasing the voltage between the electrodes for example).

Of course, this is of no consequence for the comparison of the signatures since the signals will all be produced according to the same operating conditions and the decrease in the intervals between peaks (inherent to the acceleration of the electrophoretic migration) should not disturb the creation of the signature.

Regarding the analysis of peak spacing for the x-coherence as defined above, the use of a sequencer such as ABI 3700 makes it not necessary to recalculate it. In the case it is necessary, it can be recalculated using the information carried by each of the partial sequences of the clones. The use of an internal migration calibration standard can be used in order to quickly obtain the signature. Alternatively, the nature of the vector, the sequence of which is known and which is located at an end of a cloned fragment can be used as a standard for the peak spacing.

Even more advantageous is the fact that sequencing using a single terminator eliminates all the problems linked to artifacts of fragment mobility in the electrophoresis capillary. This problem is crucial in a conventional sequencing reaction, in which, for the same clone, fragments which are terminated by four different terminators, and the mobility of which cannot be synchronized in a completely satisfactory manner (or even worse when the migration is accelerated), are combined.

The comparison between signatures is carried out according to algorithms that have been described above and that have been shown to be effective. The comparison between pseudo-sequences (alphanumerical signatures) can be carried out using known pieces of software such as Blast or LocalAlign, in particular with the penalty matrix as described above.

The clone analysis can be carried out on any type of library created from mRNAs derived from biological materials taken from diverse animal or plant species. It is, in fact, not necessary to have reference libraries in order to be able to analyse the genes expressed in a sample and to deduce, by comparison between libraries, differential expression profiles. After comparison of the signatures, it is possible to assign an identity to the clones by sequencing the clone located. In the case of the SAGE method, a clone can be identified from its signature only if the corresponding cDNA has indeed previously been sequenced and if the sequence is available in a reference library.

Flexibility of the method: the reading length can be extended as needed, and the duration of electrophoresis can be modulated. It is also possible to envisage sequencing the clones via one or other of the ends (3′ and/or 5′ ends). This possibility offers strategic flexibility depending on the library created, makes it possible to validate the results which are obtained by sequencing from a single end, and allows sequenced variants to be located.

This method may be used to determine which genes are expressed in a cell or a tissue, and at what level, and to study differential expression.

The invention thus relates to a method for analyzing the differences of gene expression between at least two samples, comprising the steps of:
performing the method of the invention on the nucleic acids present in said at least two samples, or on nucleic acids obtained from the nucleic acids present in said samples (in particular cDNA obtained from mRNA)
determining the number of occurrences of a given signature in each sample, and
comparing the number of occurrences of said given signature between said samples,
relating the difference observed in the level of occurrences said given signature to the differential expression of the genes leading to said signature, between said at least two samples.

This method can be perfected by including a step of normalization of the number of occurrences of the signatures versus an internal standard (such as actin), in order to get a quantitative result for each sample, rather than a comparative result between samples.

In the most preferred embodiment, said samples are cDNA libraries obtained from mRNA from two samples (cells, tissues . . . ) submitted to different conditions, in particular chosen in the group consisting of sick/healthy, tumoral/non tumoral, difference of stress, difference of tissues . . . .

Indeed, comparison between libraries makes it possible to define, for the same tissue or the same cell type, the variations in the expression levels for various genes (without any limitation regarding the number of genes studied and without, a priori, any limitation regarding the quality of the genes themselves), for different growth, environmental, physiological or pathological conditions.

It is firstly aimed to use DACS for clonal selection (see also the methods of use). This selection makes it possible to create supports (slides, microarrays) adapted for a thorough study of a differential condition (sick/healthy, difference of tissues, difference in stress, etc).

The specificity of DACS signatures being very high, whatever the size of the genome, a few transcripts of the same gene is sufficient to identify the gene as an expressed gene. The identification against the reference, if it exists, is more deterministic than for SAGE, due to this specificity (longer signature). If there is no reference, DACS makes it possible to carry out a complete sequence of a clone (which remains intact and easily identifiable) and to put forth an assumption on the function of the gene in question by search for homology.

A clone selected because of its differential expression between two conditions, could then be also used as a product for PCR, and also as a primer matrix, with the certainty that it indeed codes for the searched gene after obtaining complete sequence of the clone. In the case of the DACS, it is not only possible to have a sequence, but also a chromatogram and data of “Phred quality” (for information, see http://www.genome. washington.edu/UWGC/analysistools/Phred.cfm, or http://www. phrap.org/), allowing to avoid the drawing of a oligo on a doubtful area.

The method makes it also possible to carry out a step for clustering and for sorting the clones in a DNA library (screening). It is, in fact, possible to observe, after sequencing multiple clones, which rare clones had, to date, remained undetected in the cDNA libraries for various cells, and result from the expression of unknown or relatively unknown genes.

The method of the invention thus allows identification of lowly expressed genes.

In the context of genome sequencing projects, the method makes it possible to characterize the redundant clones and therefore to limit the sequencing analysis to unique clones.

Thus, the invention also relates to a method for sequencing a large DNA fragment (a DNA piece comprising a few hundred kilobases to a few megabases) comprising
a) performing a random shotgun sequencing method on said DNA, fragmented and cloned within a library, or generating a library after restriction digestion of said DNA fragment, for example partial digestion with Sau3A,
b) performing the method of partial sequencing as described above on a clone in the library, in order to obtain a signature for said clone,
c) comparing said signature for said clone to the theoretical signatures of the contigs assemblies in progress, to determine if said signature for said clone is fully represented within said theoretical signatures,
d) sequencing said clone if the answer obtained in step c) is negative,
e) starting the method over from step b) on another clone in the library if the answer in step c) is positive.

Step a) is optional, and the method of the invention may be performed only on some clones after the initial “shotgun” program.

By “fully represented”, it is meant that the whole signature is found in the signature of reference. When only part of the signature is present in the signature of reference, it may mean that the clone was a chimera, or most probably that the sequence of clone constitutes an extension of an existing contig.

In a preferred embodiment, said large DNA fragment is a genome, in particular a bacterial, or a eukaryotic, chromosomal genome, or a large plasmid, or an organelle genome.

Indeed, after multiple clones have been sequenced and the sequences have been assembled, there always remain, in a genome sequencing project, a certain number of regions which are not sequenced. Sequencing these regions makes it possible to obtain the complete sequence of the genome; this step is called “finishing”. The method described here integrates into this finishing step since it makes it possible to recognize, in a genomic DNA library, the clones which contain unknown genomic fragments. It is therefore possible to readily select these clones and to concentrate on sequencing the fragments which it contains and which correspond to the genomic regions which had escaped the first sequencing phases.

The so-called “random shotgun” sequencing method proved reliable and feasible for sequencing a large number of species. Its cost remains high because of a lack of effectiveness, related to the practical limitation of the random nature of the approach.

It will be recalled here that, in the random shotgun method, the genome is randomly sheared into 500-2000 bases fragments, which are cloned into a vector, such as to create a library. Sequencing of the clones is then performed, with a step of assembly between sequenced clones to create the contigs. Nevertheless, it is noted that some parts of the genome are under represented in the library, and that it is not always possible to obtain the desired coverage of some parts of the genome (it is liked to have about 5 times coverage of each area, with sequencing from both strands of DNA).

Indeed, if one wishes to increase the coverage or to close gaps in an assembly, the addition of new clones from random shotgun library can prove very ineffective. Certain areas of the genome can be difficult, even impossible to clone, which is the case of genes of a species which are toxic for the bacterium host. The only alternative today is to enter in a phase of “finishing”, which is very labor intensive and consumes human expertise.

After an initial shotgun sequencing, DACS potentially makes it possible to connect a second high throughput phase, when the progression of the sequencing project decreases. In this step, DACS signatures are compared against the contigs assembly in progress, to determine if the corresponding clones would bring new information. If this is the case, then traditional sequencing is carried out, the assembly is updated, and a new cycle is started after comparison of the new DACS signatures with the updated assembled contigs. In term of new information, one can distinguish in particular:
a sequence still completely unknown (this one will probably constitute a “singleton” for the N+1 assembly)
a sequence known for a first part, unknown for a second part (this one will probably constitute an extension of an existing contig of assembly Nfor the N+1 assembly)
a known sequence being in an area of insufficient coverage compared to an objective (this one will reinforce the N+1 assembly, either by confirming assembly N, or by bringing an indication of the existence of an inconsistency in assembly N.

It is reminded that 4 clones can be analyzed at the same time in this second high throughput phase, that the sequence reaction can be limited to the desired length of DNA (up to 200 bases), when the full sequence reaction goes usually up to more than 500 bases, and that use of sequential capillary electrophoresis allows high speed analysis.

The creation of a signature which is associated with a clone and which provides the identification thereof may be of use in any domain in which the identification of DNA fragments makes it possible to trace samples or to carry out species identifications, or even to analyze relationships between individuals (agro foods, medico-legal analyses).

In the field of comparative genomics, having the complete sequence of the genome of a strain of reference, DACS makes it possible to isolate a group of clones containing a different piece of genomic DNA (new or homologous) from one or more strains of interest. The sequencing of these selected clones will make it possible to assemble new DNA and to map the partial sequences of these clones on the sequence of the whole genome of the strain of reference. The homologous DNA can directly be mapped on the reference with the help of a traditional search for homology like the Blast software tool.

Thus, the invention also relates to a method for identifying genomic differences between a first organism, the genomic sequence of which is known, and a second organism, the genomic sequence of which is unknown, comprising
a) fragmenting and cloning genomic DNA of said second organism in a library
b) performing the method of partial sequencing according to the invention on a clone in the library, in order to obtain a signature for said clone
c) comparing said signature for said clone to the theoretical genomic signature of said first organism, to determine if said signature for said clone is fully represented within said theoretical signature. (alternatively, the signature obtained in step b) can be compared to the genuine signature obtained by partial sequencing of genomic DNA fragments of the first organism.)
d) deducing the presence of a difference between said second organism and said first organism, when said signature for said clone is at least not fully represented within said theoretical or genuine signature, and optionally
e) sequencing said clone to characterize said difference.

By “fully represented”, it is meant that the whole signature is found in the signature of reference. When only part of the signature is found in the signature of reference, this usually means that the differential part is locus of genetic difference.

The generation of a signature can also provide a means for identification of discrete sequence changes and also to detect gene transcription diversity.

Indeed, carrying out a partial sequencing reaction over a reading length greater than 200 bases or over several sites of the insert simultaneously, the probabilities of being able to detect variant forms of the transcripts are increased (whether these are alternative transcripts or even discrete base modifications, mutations or polymorphisms without pathological consequences, SNP). Thus, specific application of the method of the invention allows a qualitative analysis of nucleic acids.

### EXAMPLES

### Example 1

After creating the DNA library, the DNA fragments, inserted into cloning vectors, are perpetuated in host bacteria cultured in vitro. A conventional experimental step of plating out on solid culture medium allows each of the clones making up the library to be isolated: “clonal selection” step.

The clonal vectors containing the DNA fragments are purified according to suitable methods available according to the state of the art. These elements are subjected to a partial sequencing reaction.

A primer (synthetic oligonucleotide) capable of hybridizing with one or more regions of the vector is used to carry out this reaction. The primer is, for example, labeled with a fluorochrome (dye-primer) (the chemical composition of the fluorochrome is chosen as a function of the range of molecules available according to the state of the art at the time and as a function of the spectral characteristics expected for this molecule which will serve as a tracer for the generation of the detectable signal: excitation length and emission wavelength). The primer elongation reaction is catalyzed enzymatically under the conditions which satisfy the principle of the “chain elongation termination sequencing” reaction described by Sanger et al (Proc Natl Acad Sci U S A. December 1977; 74 (12): 5463-7). Succinctly the reaction medium contains the natural nucleotides required for extension of the primer by the enzyme, the enzyme (DNA-dependent DNA polymerase) and a terminator. The terminator is a nucleotide analogue which prevents any subsequent nucleotide polymerization and therefore interrupts the elongation of the DNA polymer being synthesized by the enzyme. In this reaction, the terminator is a dideoxynucleotide. Dideoxy C or dideoxy G or dideoxy A can be used alternately, but the sequencing reaction does not contain all four terminator nucleotides (NB, due to the existence of sequence segments consisting of A-base repetition at the 3′ terminal end of the cDNAs, in this peculiar case use of dideoxy T, which is the nucleotide analogue incorporated by the polymerase into the DNA polymers during elongation by complementarity to the base A, is not desirable). A single sequencing reaction is carried out per clone, with a single terminator. Depending on the enzyme used, the sequencing reaction may be carried out isothermally or by repeating several steps performed at different temperatures (thermal cycling).

Conventionally, when the intention is to decipher the sequence of a DNA clone by using sequencing reactions which employ fluorescent primers, it is necessary to carry out 4 different sequence reactions with 4 fluorescent primers, and each of the reactions is carried out in the presence of a single terminator nucleotide. The use of terminators specific for the bases A, G, C or T makes it possible to obtain all possible combinations of chain elongation interruption and to deduce the sequence of the fragment after electrophoresis of the reaction products.

In the method of the invention, carrying out the sequencing reactions with a single terminator does not therefore make it possible to fully define the sequence of a DNA fragment. The reaction carried out with the dideoxy C terminator, for example, results in an electropherogram composed of peaks which reflect the incorporation of the dideoxy C into the extended DNA chains. The information regarding the A, G and T-base composition is therefore “cut” from the signal. However, the nature of the signals recorded and also the distance separating each peak (absence of signal corresponding to the DNA regions containing bases A, G or T) are two additional parameters which enrich the electropherogram of the clone sequence with dideoxy C.

The concentration of dideoxynucleotide can be adjusted in the reaction medium so as to prevent any elongation of the DNA chains beyond 200 bases.

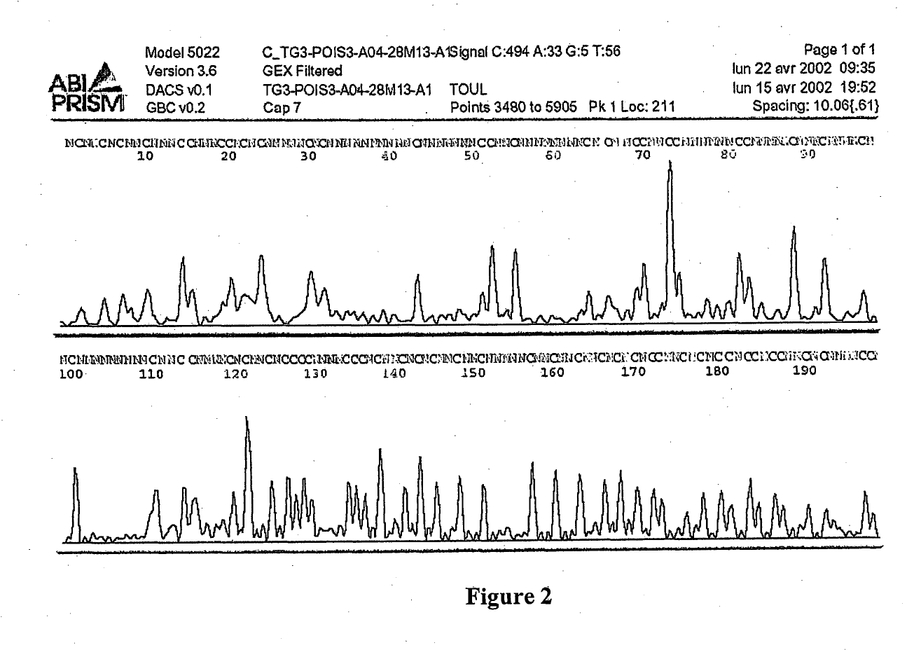

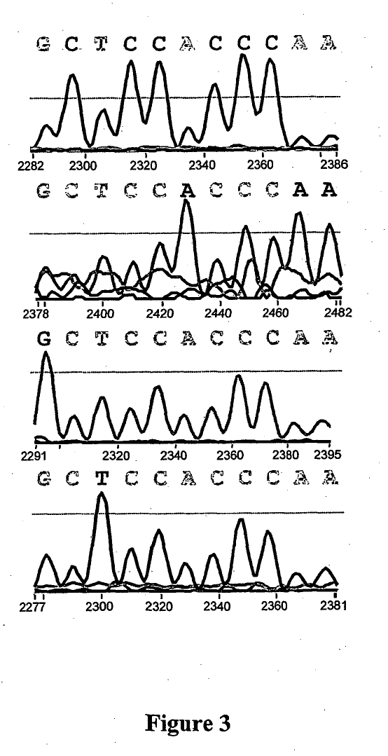

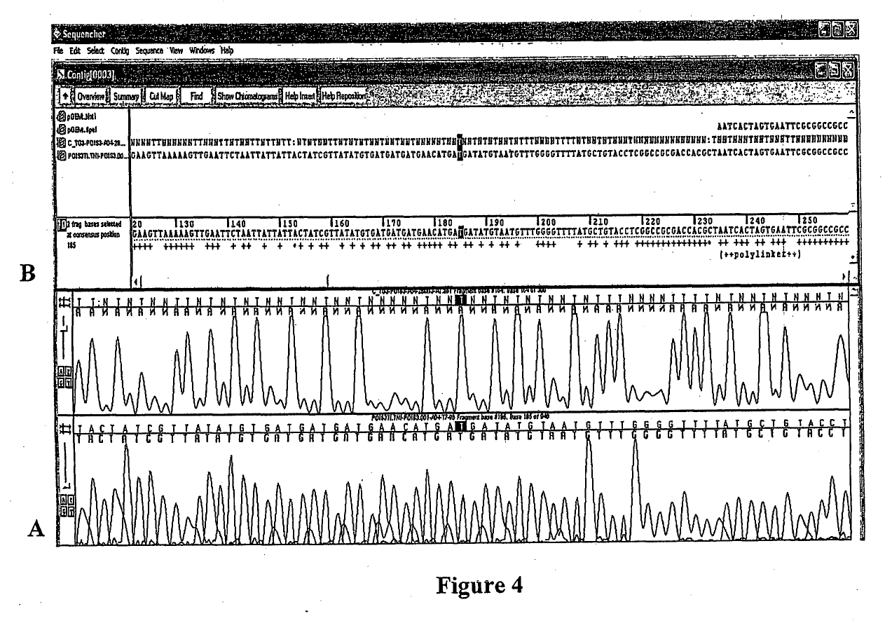

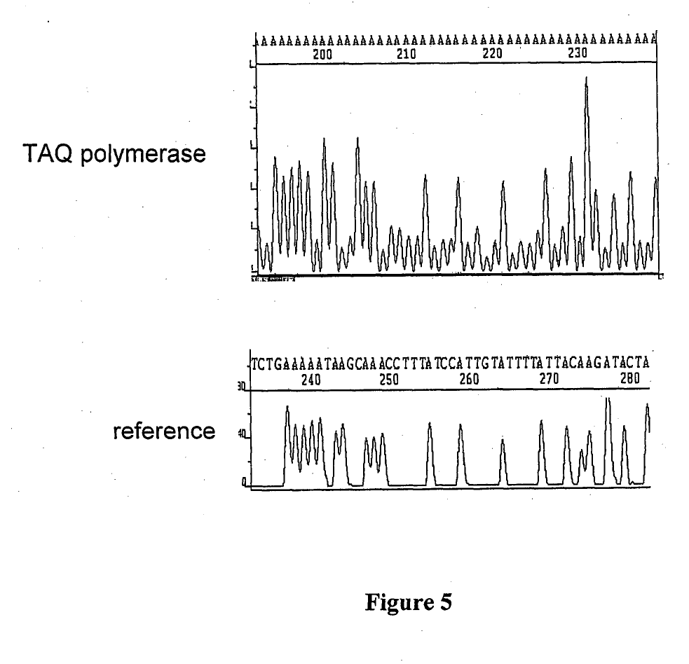

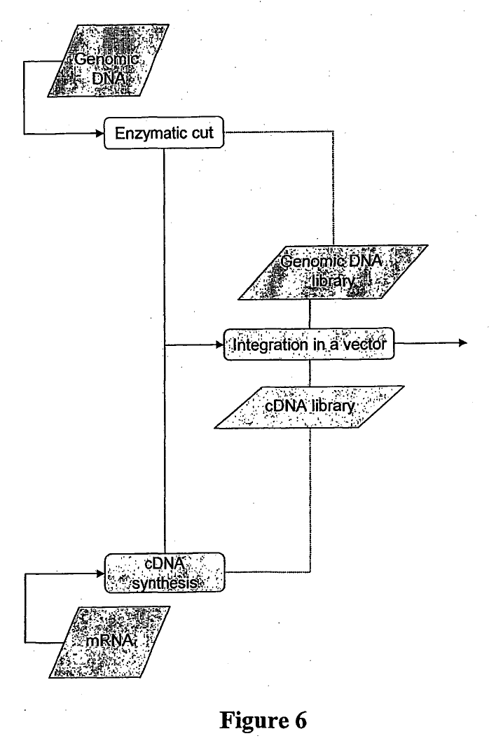

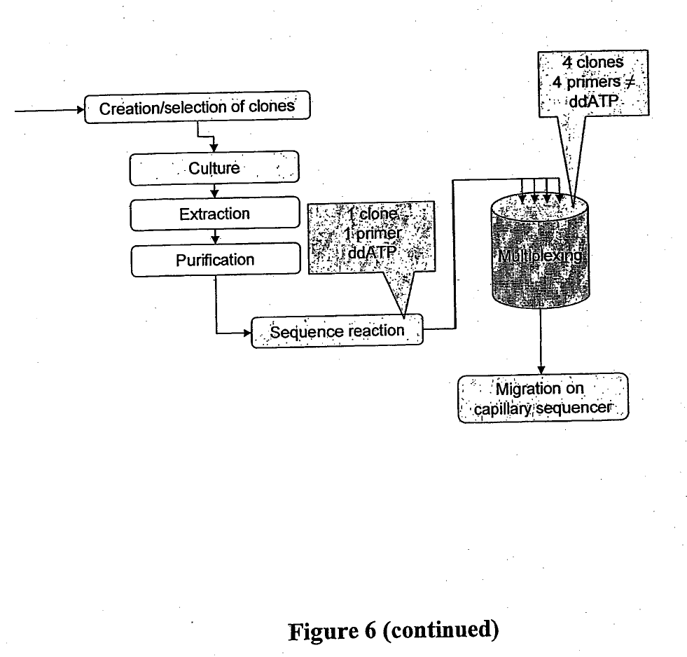

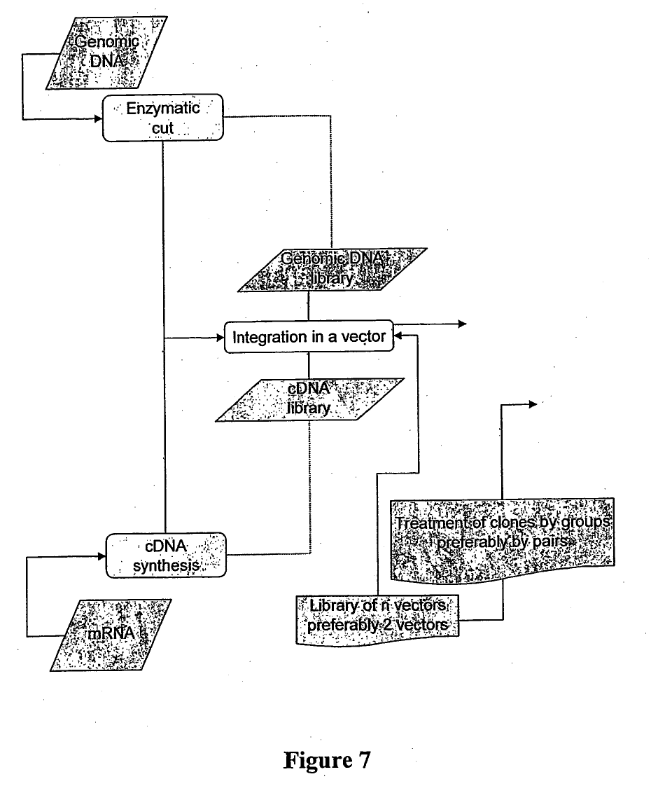

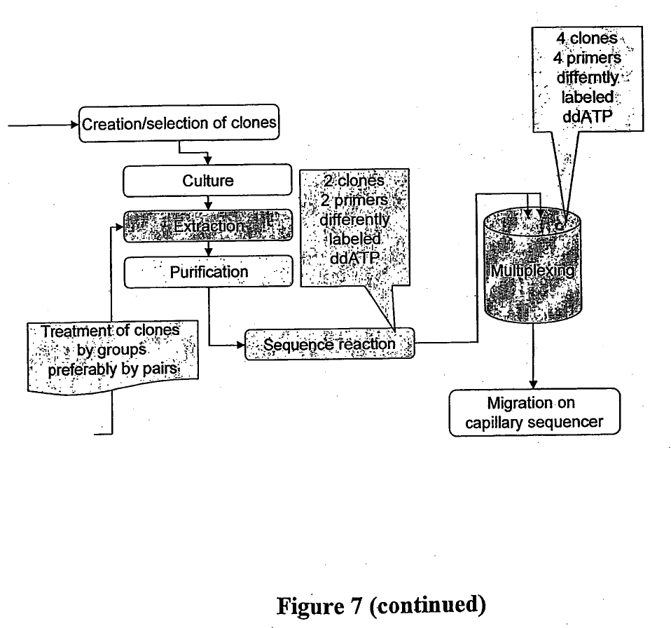

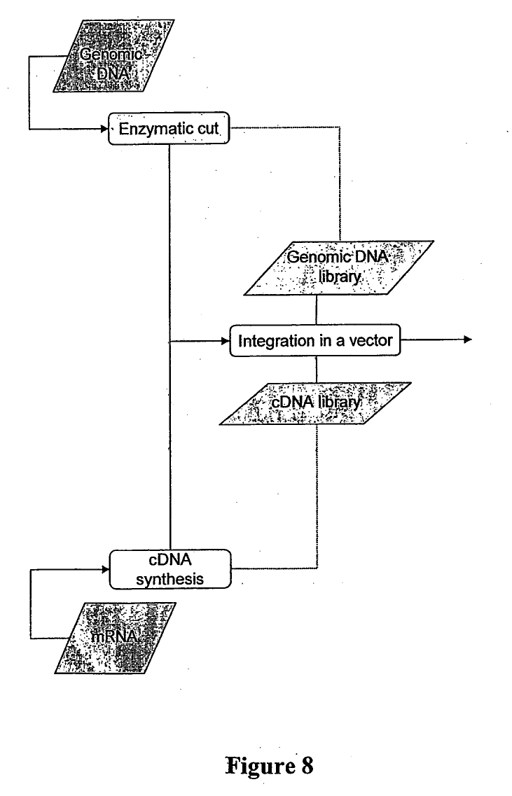

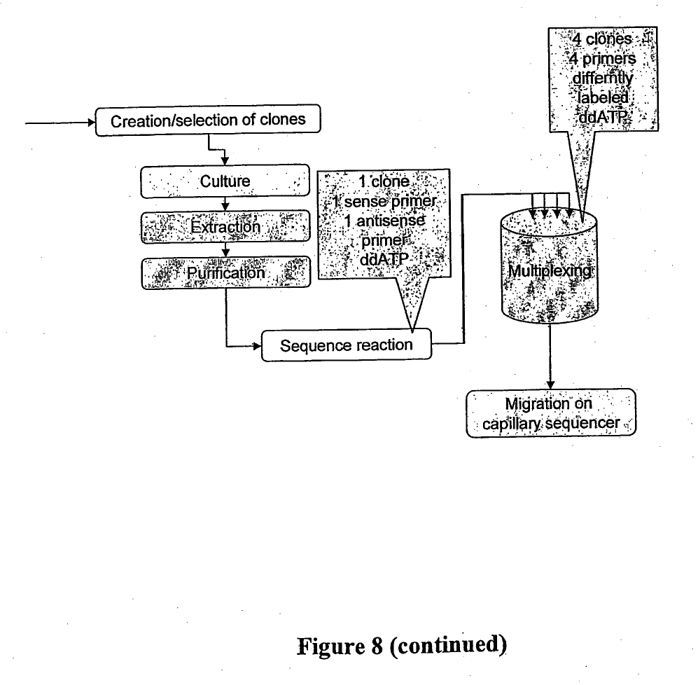

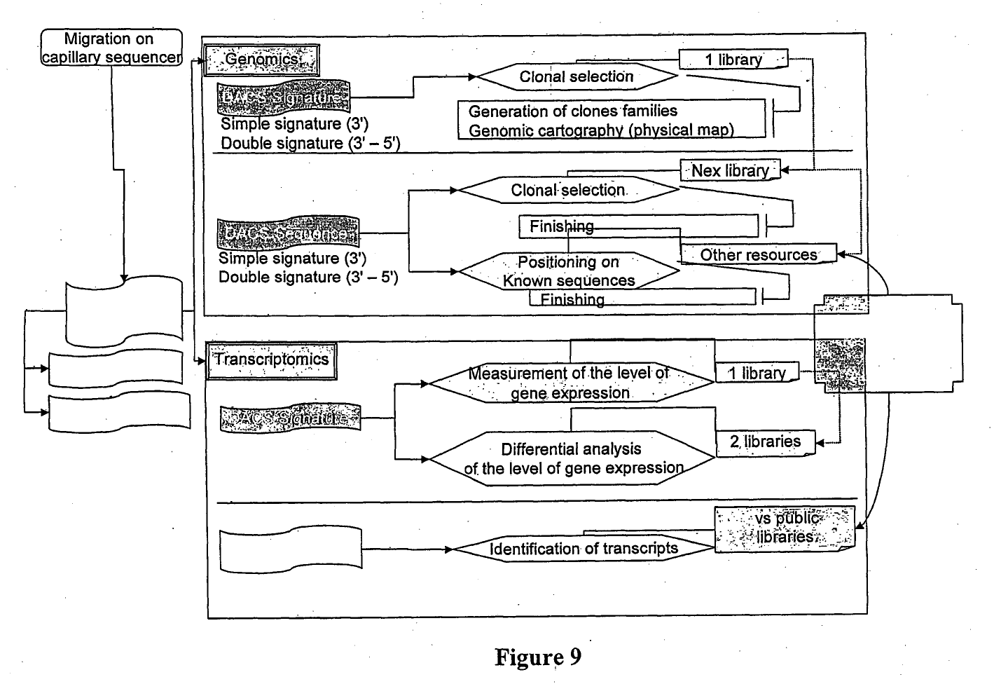

An example of a typical (single-clone) electropherogram is shown in FIG. 2.

The use of a set of 4 different fluorescent primers makes it possible to carry out 4 different sequence reactions with 4 different clones (the reaction medium contains, in each of the 4 reactions, a single dideoxynucleotide).

After these 4 sequence reactions (carried out with 4 different fluorescent primers) have been carried out, the products obtained are mixed and, optionally, desalinized and concentrated. The mixture is analyzed by capillary electrophoresis using sequencing machines.

As an alternative, the fluorescent compound which serves as a tracer may be coupled to the chain elongation terminator and no longer to the primer which constitutes the sequencing primer.

The experimental data recorded in an electropherogram therefore represent complex information which combines the partial sequencing data from 4 different clones. Analysis of the signals is therefore required in order to extract the signatures which will be used to explore the quality of the DNA library. The method described in this document presents a certain number of technical solutions, and reply in a suitable manner to the questions addressed during the validation of this method.

### Example 2

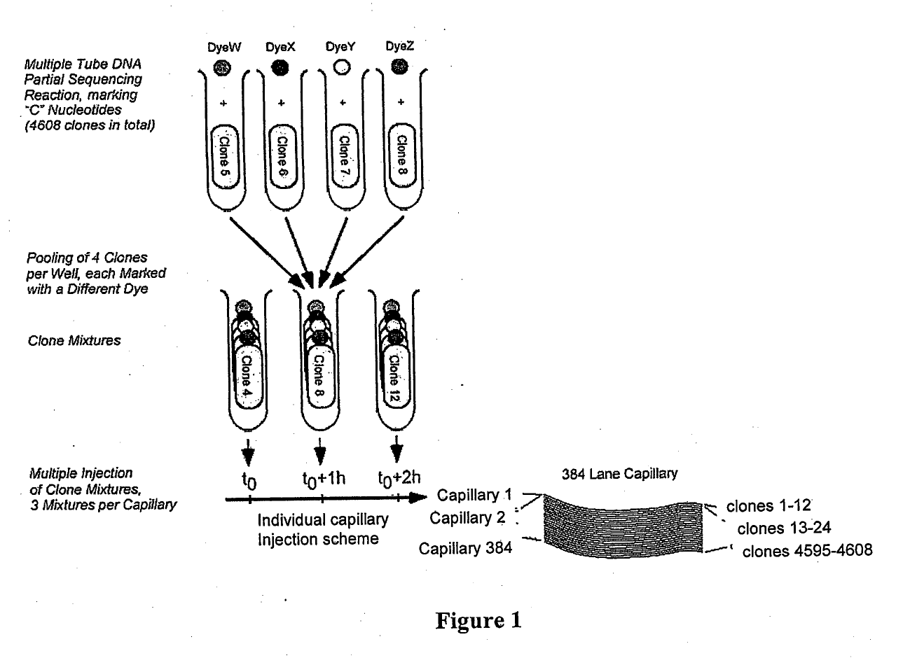

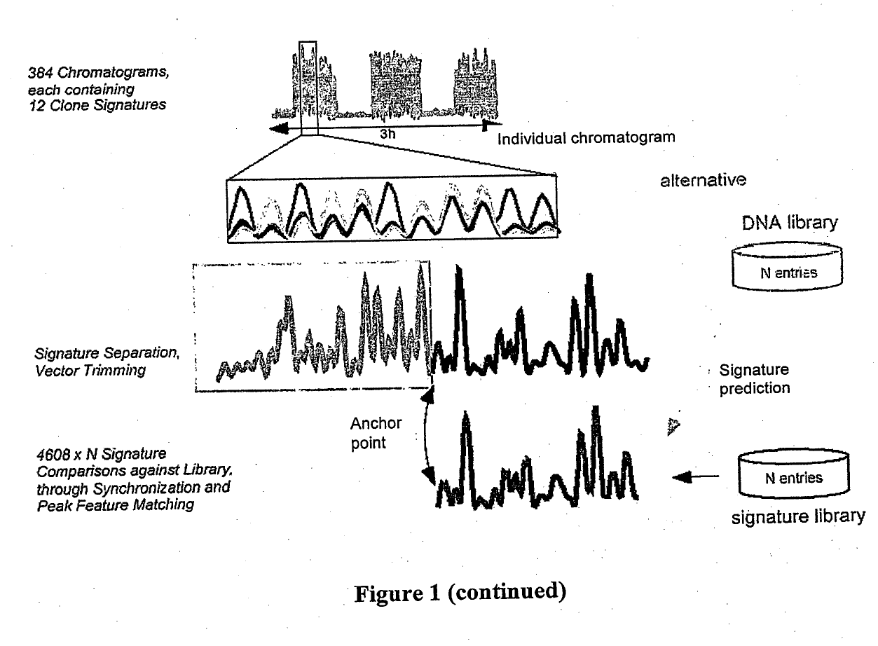

In the first instance, the signals which make up the electropherograms recorded are isolated, on the basis of the spectral properties of these signals, as 4 different electropherograms. The sequence data are then analyzed in order to locate, in the electropherogram peaks, the peaks corresponding to the bases identified which are part of the DNA fragment cloned or of a portion of the vector which was used to clone this fragment. Thus, the analysis can be restricted to only the region of interest consisting of the fragment cloned (FIG. 1, vector trimming). In addition, this step makes it possible to locate the beginning of the fragment which constitutes an anchorage point for the comparison analysis (priming site). The signals of two different clones will therefore be readily characterized by the fact that they show two peak domains: a series of identical peaks (corresponding to the sequence of the vector) before the priming site and a series of non-homologous peaks beyond the priming site.

As is indicated above, the clones are analyzed by partial sequencing, which, to a first approximation, consists in generating a sequence “depleted” of ¾ of the sequence information. To analyze the electropherogram with the accuracy required in order to validate the position of the peaks corresponding to the incorporation of the chain terminator and to guarantee perfect estimation of the number of bases separating each of the peaks of the electropherogram, a calibration of the retention times of the fragments subjected to the electrophoresis, as a function of their size, might be needed is certain cases so as to have a so-called x-coherence of the signal. This calibration can be based on a measurement of the distance separating the peaks which correspond to fragments the size of which differs by a single base (determining peak spacing). This calibration can be necessary when the distance separating two peaks corresponding to fragments the size of which differs by a single base is greater at the end of electrophoresis than at the beginning of electrophoresis. Calibrating peak spacing on the electropherogram also offers the advantage of greatly facilitating the process of comparing electropherograms to one another. Alternatively, with a machine such as ABI 3700, no calibration of peak spacing is necessary, due to the very good x-coherence (i. e. good regularity of the peak spacing) obtained with this particular DNA Sequencer in the specific size of window selected for the analysis (i. e. fragment size preferably ranging from 10 to 300 bp).

When needed, this calibration, by ensuring peak synchronization, considerably increases the validity of the inter-electropherogram comparison. Two technical possibilities have been envisaged for carrying out this calibration of peak spacing. Firstly, it is possible, during the electrophoresis, to migrate a standard composed of a range of fragments having calibrated and evenly distributed sizes. During the analysis, it is the distances separating the peaks of the standard sample which are measured and are used as a reference to calibrate the peak spacing in the clone analysis electropherograms. This solution is satisfactory but, nevertheless, complicates all the possibilities for recording the spectral data during the electrophoresis. Another solution can be preferred. This solution is based on the analyzis of the frequency of appearance of the peaks during electrophoresis through a Fast Fourier transform method, which can generate a diagram showing the evolution of the frequency of detection of the signals as a function of the progress of the electrophoresis (as a reminder, the signals recorded at the start of electrophoresis correspond to fragments small in size composed of a few bases, those detected later are associated with fragments larger in size: several tens, or even hundreds, of bases). An additional advantage of this method lies in the fact that it can also allow the background noise to be filtered out and therefore make it possible to disregard a certain number of parasitic peaks which are not authentically associated with bases corresponding to an incorporation of the terminator. The peak spacing can therefore be estimated with accuracy over the entire length of the electropherogram. Consequently, the electropherograms can be standardized with a uniform peak spacing over their entire length. Thus, peak number counting is facilitated and the number of other bases, which corresponds to the distance which separates each main peak, is determined with an accuracy so far unequalled.

The signature may then be generated.

### Example 3

The simulations which we have performed, in silico, show that the signals recorded for a partial sequencing reaction covering a length of 80 to 100 base pairs for human cDNAs corresponding to transcripts of genes from chromosome 22 constitute unique signatures which allow reliable identification of the cDNAs in a complex human cDNA library. This evaluation has made it possible to define that the generation of fluorochrome-labeled DNA fragments shorter in length than about 200 bases in the partial sequence reactions is sufficient to allow identification of the different signatures. The operating conditions for the sequence reaction have been optimized accordingly. Another way to control the length of the fragment consists in adequately controlling their sizes during their preparation (for example fragments generated by PCR or by restriction digest). In addition, 200-base fragments can be analyzed in a few tens of minutes using an electrophoresis machine. We have therefore taken advantage of this observation by carrying out the electrophoresis of several samples in a paced manner. This paced analysis consists in successively injecting several different samples into the same electrophoresis capillary.

Thus, without having to regenerate the capillary between each injection, it is possible to carry out several sample injections by staggering the injection times such that the analysis of the first sample is finished at the moment the following one is injected, and so on. This manoeuvre (multiple injection or multiloading) considerably increases the overall yield of the method in terms of the number of clones analysed per day and per machine, and therefore leads to a very large decrease in costs. As things stand in terms of electrophoresis machines and the performance of the chemical polymers which fill the analytical capillaries, it is possible to envisage performing 3 successive injections for the same capillary. This leads to 3 samples being analysed in approximately 1.5-2 hours per capillary.

The processing of the electropherograms therefore allows a clone-specific signature to be created, which combines the following 3 elements: number of peaks corresponding to the incorporation of the terminator, distance separating the peaks, which can be expressed as number of bases, signal intensity relative to background noise (height of the electropherogram peaks). It is possible to add to these three signature-defining parameters a fourth element which is intended to take into account the modifications of the structural characteristics and position of the peaks as a function of the environment of the sequence (contextual effect linked to the sequence of the clone in the area of the region in which the terminator has been incorporated). This predictive and intelligent analysis of the signatures is carried out by comparison to libraries of artifacts of terminating incorporation and of electrophoretic migration of fragments which have already been listed.

### Example 4

The relevant “biological” information regarding the libraries studied is obtained by comparing the signatures to one another. This comparison is based on signal synchronization, comparison of the signature-defining parameters. From a technical point of view, these operations are managed by algorithms which have been developed and are described in U.S. Pat. No. 6,195,449, the content of which is incorporated herein by reference.

The comparisons between signatures may be produced according to two methods. In the first method, only the signatures obtained during the partial sequencing of the clones of a library are compared to one another. At the end of this analysis, it is possible to determine the clones which are identical over the region covered by the signature and to accurately quantify the representation of each of these clones in the library (this approach makes it possible to reply to the following question: how many times is the same signature—and therefore the same clone—found in all the signatures of the library?). For this exploration, the biological identity of the clone is not required, a priori, to deduce the quantitative information relative to the representation of the clones. However, after quantitative exploration of signature representation, it is possible that this may lead to a desire to know which genes have produced the signatures which are of interest to the research scientist.

In the case of cDNA libraries, the signatures of interest are selected by the biologist as a function of the quantification results for the signatures of a DNA library created from mRNAs extracted from a single tissue or from a single cell type; or else by comparison between the data from quantifying the signatures in 2 libraries created from mRNAs extracted from cells subjected to different growth conditions or having different physiological or pathological states. In this approach, the intention is to find out the genes for which the rate of expression varies as a function of various parameters. The clones of interest can be completely identified by their signatures and are already listed and classified in the library with all the other purified clones. As a result, it is very easy to select these clones and subject them to thorough analysis by complete sequencing of the cloned DNA fragment.

In the case of genomic DNA libraries, qualitative comparison of the signatures of the clones making up this library makes it possible to recognize the clones which are identical over the region covered by the signature, and therefore to propose an important strategic aid for choosing the clones of interest in the context of a project for sequencing a complete genome (or part of a genome). In fact, after having created the library of genomic DNA fragments, it is advantageous to locate the different clones and to concentrate the sequencing work on these clones only. In this way, the sequencing of a genome is optimized by avoiding sequencing over- represented clones, which would constitute a superfluous redundancy of information for the project.

The second method of comparison consists in comparing the signatures obtained experimentally to the known sequences of clones in the databases (reference libraries). In this scenario, the information from partial sequencing (presence of base revealed by the peaks of the electropherogram and distance between the peaks) consists of two pieces of data which make it possible to search for homologies between these signatures and the existing sequences of known genes or cDNAs. By carrying out this comparison, it is possible to directly associate a biological identity with the signature. To increase the probability of the prediction which can be made by the homology search, it is possible to create reference libraries, in silico, which will contain signatures deduced from the known sequence of the clones. The signatures which will be in silico generated in this way may take into account the contextual effect of the incorporation of terminators mentioned above. The synthesis, in silico, of signatures may also take into account the specificities linked to the sequencing reaction conditions (type of primer, type of terminator, nature of the enzyme, characteristics specific to the electrophoresis conditions: duration, voltage, capillary-filling polymer, optical detection, etc. ). In all cases, of course, subsequent to this first analysis, the thorough sequencing of a clone of interest present in the library will provide a peremptory response regarding the identity of the clone.

### Example 5

### Methods of Use

5.1. Analysis of the Transcriptional Profile of a Cellular Type

Principle: polyA messenger RNAs of a eukaryotic cell are converted in cDNA and individually kept in the clones of a library of cDNA. A quantitative yield on production of the cDNA is expected so that the frequency of representation of each of these cDNA within the library is an indicator of the level of expression of each of the genes within the analyzed cell (i.e., the stronger a gene is expressed in a cell, the larger is the number of representative clones of this gene in the library).

Aim: The biologist wishes to have a measure of the rate of expression of each of the genes of a cell. He can so discover which are the genes the most (or the least) constitutively expressed, in a cell.

The allocation of an identity and a biological function to each of the listed clones, can be made at once, on condition that the genes or EST from the organism from which the cells aroused are already known.

Should the opposite occur, the clones of interest are then extensively sequenced. Therefore, an allocation of function “by proximity” can be then predicted by comparing obtained sequences with those of the genes known for close species which are inventoried in databases.

Interest: The biologist thus reaches a multiparametered datum which is characteristic of the analyzed cellular type. He can detect the genes that are specifically expressed in a type of cell, organ or species.

Practice: This analysis requires the creation of a DACS library from polyA RNA. The extraction of reliable quantitative data requires the production from 10 to 40 000 signatures by libraries.

The mode of analysis of signatures is made by:
comparison between signatures,
comparison with resources of known sequences.

The analysis can be followed by:
the extensive sequencing of the most expressed clones,
the search for modifications in similar transcripts and signing the possible existence of alternative transcripts,
the search for function by bio-computer analysis by means of data bases,
the production of titrated and purified PCR fragments for the manufacture of arrays,
the creation of kit of RT-PCR for quantitative analysis of the transcripts of interest.
   5.2. Comparative Analysis of the Transcriptional Profile of Two Cellular Types

Principle: PolyA messenger RNAs of two or several types of eukaryotes cells are converted in cDNA and individually kept in the clones of a library of cDNA. This analysis requires the creation of so many libraries of cDNA whom there is of different cellular types. For each of the libraries, the frequency of representation of the cDNAs is an indicator of the level of expression of the various genes within the analyzed cell. The comparison of the quantitative data obtained from a library to the other one makes it possible to discover the differences of profiles of expression between various cellular types.

Aim: The biologist wishes to have a measure of the level of expression of each of the genes of a cell. He can so discover which are the genes over-or under-expressed in a cellular type with regard to the other one.

The allocation of an identity and a biological function in differentially expressed clones can be made at once by comparison with databases or after sequencing of clones.

Interest: The biologist can compare the profiles of expression of the genes in cells. He can thus study the impact of physiological or pathological events as well as the effect of various stress or the influence of a molecule on the expression of the genes of a cell or a tissue.

He therefore can connect a phenotype with a variation of the level of expression of one or several genes.

He can determine the effect of toxicity of molecules; observe the activation of metabolic pathways or particular genes; analyze descriptive intracellular signaling; analyze the adaptive response of a cell to a specific environmental situation, to a microbial invasion.

Realization: This analysis requires the creation of a minimum of 2 DACS libraries from polyA RNA resulting from at least two cellular types, or from the same cellular type, from two organisms. The extraction of reliable quantitative data requires the production from 10 to 40000 signatures by libraries.

The mode of analysis of signatures is made by:
comparison between signatures,
comparison with resources of known sequences

The analysis can be followed by:
the extensive sequencing of differentially expressed clones,
the search for modifications in similar transcripts and signing the existence of alternative transcripts,
the search for function by bio-computer analysis by means of data bases,
the production of titrated and purified PCR fragments for the manufacture of arrays,
the creation of kits of RT-PCR for quantitative analysis of the transcripts of interest.
   5.3. Screening of Clone Libraries and Finishing of Genome Sequencing

Principle: The genomic DNA in the course of analysis is split up in a multitude of elements kept in a library of clones. The comparison of DACS signatures makes it possible to make a sorting of clones to keep only those which present a major interest within the framework of a program of complete sequencing of the genome of an organism (bacterium for example).

Objective: the biologist wishes to track down in a library of clones which are the clones which contain fragments of genome corresponding to regions of the genome for which he wishes to make a detailed sequencing.

Interest: The biologist can isolate the genomic clones of sufficient size which will allow him to focus on genomic regions not yet analyzed by sequencing.

In particular, having realized the “sequencing draft” of a genome, the researcher is brought to look for clones containing the fragments of genomic DNA covering regions not yet sequenced. He can so abandon clones containing fragments the sequence of which was already clarified.

Realization: this analysis requires the creation of a library of clones containing fragments of genomic DNA. Clones can be obtained by mechanical fragmentation of the genomic DNA or by partial enzymatic digestion. The number of signatures to be produced by library depends mainly on the size of the genome of the studied organism. The obtained DACS signature is compared by alignment of the alphabetic signatures with the sequences of contigs or already analyzed regions. Only the unique clones (i.e. clones having a signature which is not an integral part of a known contig or analyzed region) are kept for detailed analysis.

The mode of analysis of signatures is made by:
comparison with resources of known sequences (such as sequence assemblies obtained during the “sequencing draft” process)

The analysis can be followed by the extensive sequencing of the selected clones.

5.4. Screening of Genomic Library

Principle: The genomic DNA has been sheared into many fragments that have been inserted into vectors. Libraries can be made in a large number of different vectors (BAC, PAC, cosmids, phages, . . . ). The size of the inserted fragments can range between 10 to more than 150 kbp according to the vector used. Fluorescent fragments are generated after individual restriction digestions of a large number of clones from the library. After separation by electrophoresis of the labeled fragments of each clone, a clone is characterized by a specific signature corresponding to the pattern made up of the different fluorescent peaks detected at the end of the capillary. These signatures are analyzed by the computer-assisted process which is used and described in this document.

Objective: The biologist wishes to find all the identical clones in a library of large DNA fragments. In addition, the biologist wishes to make a scaffold of different clones that may ideally cover the whole genome sequence. This scaffolding is performed by detecting all the clones that contain partially overlapping labeled fragments.

Interest: The biologist can isolate the appropriate (ideally, the minimum) number of clones with insert sequences that can, after assembly, cover all the genome of interest to be sequenced.

Realization: this analysis requires the creation of a library of clones containing large fragments of the genomic DNA. Individual clones are digested by restriction enzymes and end-labelled with fluorochromes by any state-of-the-art means. The signatures are produced by electrophoresis of the resulting mixture and peak detection. The signatures obtained are compared by alignment of all the different clone signatures. Identical fragments of the clones can then be identified and overlapping between the various clones can be inferred from this comparison. A scaffold of all the different clones can be generated and is representative of the total physical map of the genome. The minimum number of clones is kept for further sequencing and to secure the sequencing of the whole genome of interest.

The mode of analysis of signatures is made by:
Comparison between all signatures
Assignment of all the identical labeled fragments between the signatures
Scaffold building of all the overlapping clones

The analysis can be followed by:
The extensive sequencing of the isolated clones
   5.5. Discovery of Specific Genomic Regions in the Genomes of Nearby Species

Principle: genomic DNA of at least two organisms of nearby species are fragmented up in elements of various sizes and kept in libraries of clones. The comparison of DACS signatures obtained on the clones of both libraries makes it possible to sort clones to keep only those that present a major interest within the framework of a program of comparison of genomes of nearby species.

Objective: the biologist wishes to have the possibility of tracking down in a library of clones which are the clones which contain DNA fragments corresponding to regions of the genome which are present in a species and not in the other one.

Interest: the biologist can thus look for the genomic regions which show macroscopic differences between species.

This search makes it possible to discover what are the original elements which are responsible for the virulence of certain bacterial strains (enzymes, synthesis of the antigenic determinants of surface, pathogenicity islets).

The search for genetic factors responsible for the appearance of specific bacterial features such as antibiotics resistance can also be envisaged.

Realization: this analysis requires the creation of at least two libraries of clones containing genomic DNA fragments. Clones can be obtained by mechanical fragmentation of the genomic DNA or better by partial enzymatic digestion. The number of signatures to be produced by library depends mainly on the size of the genomes of the organisms to be studied. The analyses of difference between the genomes are made by comparison of all the DACS signatures obtained for each of the 2 genomes, or by comparison of DACS signatures obtained for a library with the sequences of the genome of the reference species.

The mode of analysis of signatures is made by
comparison between signatures,
comparison with resources of known sequences

The analysis can be followed by:
the extensive sequencing of the isolated clones,
the search for function by bio-computer analysis by means of data bases,
the production of titrated and purified PCR fragments, probes or oligonucleotides for the manufacture of arrays or diagnostic kits.
   5.6. Screening of Libraries or Sub-Libraries of cDNA Clones

Principle: the clones of a whole library of cDNA are screened by phenotypic analysis by means for example of the technique of the double-hybrid so as to generate a sub-library of cDNA of interest that give positive results in the assay used. This technique is used to reveal interactions between proteins. Alternatively, sub-libraries of cDNA of interest can be created by subtractive approaches so as to reduce the complexity of the library and to select clones related to specific biological phenotypes or events. Subtraction can be performed either by hybridization of clones or probes or by affinity capture or any other means. This clone selection makes it possible to put in evidence several thousand clones of interest. The DACS analysis makes it possible to sort out clones to direct the investigation to the most interesting clones.

Objective: the biologist wishes to have a method which allows him to recognize quickly and economically which are the identical clones in a library (creation of categories of clones of the same nature)

Interest: the biologist can isolate from every group consisting of identical clones, one representative clone to be analyzed in greater detail.

This approach can be used for the other situations where it is tried to identify all the unique categories of clones.

Realization: the number of signatures to be produced depends on the number of clones selected thanks to the step of phenotypic screening or subtractive screening. Only the representative clones of a category of identical clones are kept for a detailed analysis.

The mode of analysis of signatures is made by:
comparison between signatures

The analysis can be followed by:
the extensive sequencing of the isolated clones,
the search for modifications in similar transcripts and signing the existence of alternative transcripts,
   5.7. Analysis of Genes Expressed in a Cell Type or Tissue

The signature of the invention may also be used in a method for analyzing the expressed genes from a cell type or a tissue, from a cDNA library obtained from total mRNA from said cell type or tissue, comprising the steps of:
a) spotting the clones of said cDNA on a solid support
b) selecting a random subset of clones in said cDNA library
c) obtaining the signature according to the invention on each clones on said random subset of step b)
d) comparing said signatures and clustering the clones according to the similarities between said signatures obtained in step c)
e) choosing and labeling the cDNA carried by the clones which are highly represented ins aid subset, (representation more than 2%)
f) hybridizing said labeled cDNA to said solid support,
g) creating a cDNA sub-library consisting of the clones for which no hybridization has been observed in step f)
h) repeating said steps b) to g) on said sub-library as long as the number of clones in said cDNA sub-library remains too high,

The starting material is a cDNA library obtained from total mRNA from said cell type or tissue. It is highly desirable that said cDNA library is obtained directly from mRNA, without introducing any bias, in order to have a representation of the expressed genes in said cell or tissue as reliable as possible.

The starting cDNA library may contain a large number of clones, and preferably more than 50,000 clones.

Step a) consists in spotting the clones on a solid support, such as a membrane, usable for dot blot.

In step b), from the starting library, one would choose a subset of clones, for example 1536 clones. This number corresponds to 4 * 384, wherein it is possible to run 384 samples at one time on capillary DNA sequencers. In a preferred embodiment multiplexing can be performed, as described above and 4 clones may be analyzed for each capillary.

The signatures of the clones obtained in step c), are analyzed and the clones having the same signatures are similar and classified together. The prevalence of the most abundant clones will be measured, as their number in the subset is statistically significant.

For example for mammalian species, about 5-10 species of superprevalent DNA comprise at least 20% of the mass of mRNA, 500-2000 species comprise4o%-60% of the mRNA mass, and 10,000-20, 000 account for <20-40% of the mRNA mass (Carninci et al, Genome Res. 2000 Oct; 10(10) :1617-30).

The most prevalent cDNA are labeled and hybridized on the solid support, (step f), making it possible to subtract said most prevalent cDNA from the initial cDNA library and obtain a sub-library of cDNA that are less expressed (step g).

Note that a quality control can be performed if hybridization occurs on a clone the signature of which has not been classified as a labeled cDNA. This clone may be more thoroughly analyzed, as its cDNA may represent a differentially spliced transcript, or present similarities with the labeled cDNA.

The method is repeated on a subset of said sub-library, for example, using 4*1536 clones.

At each step, it is possible to detect the cDNA corresponding to mRNA that are expressed at about the same level. Note that the subtraction step makes it possible to create a cDNA sub-library enriched in rarer cDNA, thus increasing the relative number in the sub-library of clones that were not abundant in the previous round, thus making significant the detected number of DACS signatures at the round of analysis.

Eventually, the full library will be studied, and it is possible to have the signature and to obtain one clone of each of the different cDNAs present in the starting library, as well as its quantity. The sorting leads to a normalized library, containing about all the different cDNA initially present in the cDNA library, but with only one clone to represent a specific cDNA.

Note that the above-described method can also be performed with full sequencing instead of partial sequencing according to the invention, and that is also an object of the invention.

The invention also relates to a method for creating a normalized cDNA library from a cell type or a tissue, comprising the steps of performing the method described above, in order to identify nearly all the different mRNAs present in said cell type or tissue, and creating said normalized library, by clustering the clones representing all expressed genes, and optionally indicating their proportion in said cell type or tissue, as well as a normalized library obtained by said method.

With the above described method, it is possible to identify cDNA that are present in about the same numbers in said cell type or tissue. It is thus possible to define a method for designing nucleic acid arrays bearing probes complementary to genes that are expressed at a similar level in a cell type or tissue, comprising the steps of:
performing the method above described, wherein said labeled cDNA at each step e) represent genes that are expressed at a similar level in said cell type or tissue,
selecting probes complementary to said labeled cDNA in each steps, and
designing said nucleic acid array by fixing said probes to a solid surface.

A nucleic acid array obtained by said method is also an object of the invention.

1. A reproducible, discriminative and x-coherent signature from a nucleic acid molecule, comprising a signature obtained by a method comprising the steps of:
a) performing an enzymatic chain elongation reaction, using at least one primer to start the elongation process, and at least one chain extension terminator, whereas not all four possible terminators are used, in order to obtain differentially sized products, wherein said differentially sized products emit a detectable signal,
b) separating the differentially sized products obtained in step a),
c) detecting the detectable signals corresponding to each of the differentially sized reaction products after separation, and
d) creating the signature directly or indirectly based on the detected signals in step c).

2. The signature of claim 1, consisting of a signature obtained by a method comprising the steps a) to d) of claim 1.

3. The signature of claim 1 or 2, wherein exactly one chain extension terminator is used.

4. The signature of claim 1 or 2, wherein exactly two chain extension terminators are used.

5. The signature of claim 1 or 2, wherein exactly three chain extension terminators are used.

6. The signature of claim 1, wherein said chain extension terminator is a dideoxyribonucleotide.

7. The signature of claim 1, wherein only one primer is used in said enzymatic chain elongation reaction.

8. The signature of claim 1, wherein more than one primer is used in said enzymatic chain elongation reaction.

9. The signature of claim 8, wherein two primers are used in said enzymatic chain elongation reaction, starting from the 3′ and 5′ ends of said nucleic acid molecule.

10. The signature of claim 1, wherein separation of said differentially sized products is performed by mass spectrometry.

11. The signature of claim 10, wherein said signature in step d) is created from a graph, drawn on the basis of the detected signals in step c).

12. The signature of claim 10, wherein said signature in step d) is created directly from the signals obtained in step c).

13. The signature of claim 12, wherein said signature comprises the intensity of said signals and/or the difference of mass between two consecutive signals.

14. The signature of claim 1, wherein separation of said differentially sized products is performed by denaturing electrophoresis.

15. The signature of claim 14, wherein said signature in step d) is created from or is an electropherogram, based on the detected signals in step c).

16. The signature of any one of claims 14 and 15, wherein said signature in step d) is created directly from the signals obtained in step c).

17. The signature of claim 16, wherein said signature comprises the intensity of said signals and/or the time lapse or fragment length in bases between two consecutive signals.

18. The signature of claim 15, wherein said electropherogram is obtained from an ABI 3700 DNA Sequencer (Perkin Elmer, Applied BioSystems, Inc).

19. The signature of claim 15, wherein said electropherogram is created with means of a computer-assisted “basecalling”.

20. The signature of claim 19, wherein said computer-assisted “basecalling” is performed with the Sequencher software (Gene Codes Corporation).

21. The signature of claim 1, wherein said enzymatic chain elongation reaction of step a) is performed on a limited number of bases of said nucleic acid molecule, for example about 200 bases.

22. The signature of claim 1, wherein said detectable signal is carried by a label on said primer.

23. The signature of claim 1, wherein said detectable signal is carried by a label on said chain extension terminator.

24. The signature of any one of claims 22 and 23, wherein said label is a fluorescent label.

25. The signature of any one of claims 22 and 23, wherein said label is a radioactive label.

26. The signature of claim 14, wherein said electrophoresis is a capillary electrophoresis.

27. The signature of claim 14, wherein said electrophoresis and signal detection steps is simultaneously performed on multiple nucleic acid molecules subjected to step a), wherein said detectable signal emitted by the chain elongation products is different for each of the multiple nucleic acid molecules.

28. The signature of claim 27, wherein said detection step comprises the step of detecting, distinguishing (demultiplexing) and recording the signals specific to each of the multiple nucleic acid molecules.

29. The signature of claim 28, wherein said demultiplexing is computer- assisted.

30. The signature of claim 1, wherein said enzymatic chain elongation reaction is performed with thermosequenase.

31. The signature of claim 1, wherein said enzymatic chain elongation reaction is performed with TAQ polymerase.

32. The signature of claim 1, consisting of a string of alphanumeric characters with a specific character for the bases corresponding to specific nucleotide identified due to the presence of the used chain extension terminators, and a “joker” sign for the other bases, wherein said method further comprises the step of allocating the alphanumeric character (s) corresponding to the bases detected by the used terminator (s), and a joker sign corresponding to the non detected bases.

33. The signature of claim 32, wherein exactly one chain extension terminator is used.

34. A signature specific for a nucleic acid molecule, consisting of an electropherogram obtained after electrophoresis of the product of an enzymatic chain elongation reaction on said nucleic acid molecule, using at least one primer to start the elongation process, and at least one chain extension terminator, whereas not all four possible terminators are used.

35. A method for analyzing the differences of gene expression between at least two samples, comprising the steps of:
a) for each of the nucleic acids present in said at least two samples, obtaining a signature in accordance with the method of claim 1,
b) determining the number of occurrences of a given signature in each sample,
c) comparing the number of occurrences of said given signature between said samples, and
d) relating the difference observed in the level of occurrences said given signature to the differential expression of the genes leading to said signature, between said at least two samples.

36. The method of claim 35, including a step of normalization of the number of occurrences of the signatures versus an internal standard (such as actin).

37. The method of claim 35, wherein said samples are cDNA libraries obtained from mRNA from two samples (cells, tissues . . . ) submitted to different conditions.

38. The method of claim 37, wherein said different conditions are selected from the group consisting of sick/healthy, tumoral/non tumoral, difference of stress, and difference of tissues.

39. A method for sequencing a large DNA, comprising:
a) performing a random shotgun sequencing method on said DNA, fragmented and cloned within a library,
b) for a clone in the library, obtaining a signature in accordance with the method of claim 1,
c) comparing said signature for said clone to the theoretical or genuine signatures of the contigs assemblies in progress, to determine if said signature for said clone is fully represented within said theoretical signatures,
d) sequencing said clone if the answer obtained in step c) is negative, and
e) starting the method over from step b) on another clone in the library if the answer in step c) is positive.

40. The method of claim 39, wherein said large DNA is a genome, in particular a bacterial, eukaryotic or chromosomal genome, or a plasmid or an organelle genome.

41. A method for identifying genomic differences between a first organism, the genomic sequence of which is known, and a second organism, the genomic sequence of which is unknown, comprising:
a) fragmenting and cloning genomic DNA of said second organism in a library,
b) for a clone in the library, obtaining a signature in accordance with method of claim 1,
c) comparing said signature for said clone to the theoretical or genuine genomic signature of said first organism, to determine if said signature for said clone is fully represented within said theoretical signature,
d) deducing the presence of a difference between said second organism and said first organism, when said signature for said clone is not fully represented within said theoretical signature, and optionally, and
e) sequencing said clone to characterize said difference.

42. A method for analyzing the expressed genes from a cell type or a tissue, from a cDNA library obtained from total mRNA from said cell type or tissue, comprising the steps of:
a) spotting the clones of said cDNA library on a solid support,
b) selecting a random subset of clones in said cDNA library,
c) on each clone on said random subset of step b), obtaining a signature in accordance with the method of claim 1,
d) comparing said signatures and clustering the clones according to the similarities between said signatures obtained in step c),
e) choosing and labeling the cDNA carried by the clones which are highly represented in said subset, (representation more than 2%),
f) hybridizing said labeled cDNA to said solid support,
g) creating a cDNA sub-library consisting of the clones for which no hybridization has been observed in stepf), and
h) repeating said steps b) to g) on said sub-library as long as the number of clones in said cDNA sub-library remains too high.

43. A method for creating a normalized cDNA library from a cell type or a tissue, comprising the steps of performing the method of claim 42, in order to identify the total mRNA present in said cell type or tissue, and creating said normalized library, by clustering the clones representing all expressed genes, and optionally indicating their proportion in said cell type or tissue.

44. A normalized library obtained by the method of claim 43.

45. A method for designing nucleic acid arrays bearing probes complementary to genes that are expressed at a similar level in a cell type or tissue, comprising the steps of:
performing the method of claim 42, wherein said labeled cDNA at each step e) represent genes that are expressed at a similar level in said cell type or tissue,
selecting probes complementary to said labeled cDNA in each steps, and
designing said nucleic acid array by fixing said probes to a solid surface.

46. A nucleic acid array obtained by the method of claim 45.

47. A method for generating graphical data representative of the similarity of first and second x-coherent analog signatures of nucleic acid molecules, comprising the steps of:
dividing each signature into a plurality of fragments,
performing a cross-correlation of each fragment of the first signature with each fragment of the second signature, respectively, so as to generate a matrix of cross-correlation values,
generating a matrix of graphical zones for display, wherein each zone has a visual property determined from a corresponding cross- correlation value, and
displaying said matrix.

48. The method of claim 47, wherein the fragments are overlapping.

49. The method of claim 47, wherein each signal comprises individual peaks, wherein each fragment contains a plurality of peaks, preferably from 10 to 20 peaks.

50. The method of claim 47, wherein each visual property is selected from a color or a grayscale level.

51. The method of claim 50, wherein a clearer color or grayscale value corresponds to a higher correlation between fragments.
